SEQUENCE LISTING (6 sequences)

SEQ ID NO: 1 - protein, 2511 aa, Homo sapiens
MEEVVIAGMSGKLPESENLQEFWDNLIGGVDMVTDDDRRWKAGLYGLPRRSGKLKDLSRFDASFFGVHPKQAHTMDPQLRLLLEVTYEAIVDGGINPDSLRGTHTGVWVGVSGSETSEALSRDPETLVGYSMVGCQRAMMANRLSFFFDFRGPSIALDTACSSSLMALQNAYQAIHSGQCPAAIVGGINVLLKPNTSVQFLRLGMLSPEGTCKAFDTAGNGYCRSEGVVAVLLTKKSLARRVYATILNAGTNTDGFKEQGVTFPSGDIQEQLIRSLYQSAGVAPESFEYIEAHGTGTKVGDPQELNGITRALCATRQEPLLIGSTKSNMGHPEPASGLAALAKVLLSLEHGLWAPNLHFHSPNPEIPALLDGRLQVVDQPLPVRGGNVGINSFGFGGSNVHIILRPNTQPPPAPAPHATLPRLLRASGRTPEAVQKLLEQGLRHSQDLAFLSMLNDIAAVPATAMPFRGYAVLGGERGGPEVQQVPAGERPLWFICSGMGTQWRGMGLSLMRLDRFRDSILRSDEAVKPFGLKVSQLLLSTDESTFDDIVHSFVSLTAIQIGLIDLLSCMGLRPDGIVGHSLGEVACGYADGCLSQEEAVLAAYWRGQCIKEAHLPPGAMAAVGLSWEECKQRCPPGVVPACHNSKDTVTISGPQAPVFEFVEQLRKEGVFAKEVRTGGMAFHSYFMEAIAPPLLQELKKVIREPKPRSARWLSTSIPEAQWHSSLARTSSAEYNVNNLVSPVLFQEALWHVPEHAVVLEIAPHALLQAVLKRGLKPSCTIIPLMKKDHRDNLEFFLAGIGRLHLSGIDANPNALFPPVEFPAPRGTPLISPLIKWDHSLAWDVPAAEDFPNGSGSPSAAIYNIDTSSESPDHYLVDHTLDGRVLFPATGYLSIVWKTLARALGLGVEQLPVVFEDVVLHQATILPKTGTVSLEVRLLEASRAFEVSENGNLVVSGKVYQWDDPDPRLFDHPESPTPNPTEPLFLAQAEVYKELRLRGYDYGPHFQGILEASLEGDSGRLLWKDNWVSFMDTMLQMSILGSAKHGLYLPTRVTAIHIDPATHRQKLYTLQDKAQVADVVVSRWLRVTVAGGVHISGLHTESAPRRQQEQQVPILEKFCFTPHTEEGCLSERAALQEELQLCKGLVQALQTKVTQQGLKMVVPGLDGAQIPRDPSQQELPRLLSAACRLQLNGNLQLELAQVLAQERPKLPEDPLLSGLLDSPALKACLDTAVENMPSLKMKVVEVLAGHGHLYSRIPGLLSPHPLLQLSYTATDRHPQALEAAQAELQQHDVAQGQWDPADPAPSALGSADLLVCNCAVAALGDPASALSNMVAALREGGFLLLHTLLRGHPLGDIVAFLTSTEPQYGQGILSQDAWESLFSRVSLRLVGLKKSFYGSTLFLCRRPTPQDSPIFLPVDDTSFRWVESLKGILADEDSSRPVWLKAINCATSGVVGLVNCLRREPGGNRLRCVLLSNLSSTSHVPEVDPGSAELQKVLQGDLVMNVYRDGAWGAFRHFLLEEDKPEEPTAHAFVSTLTRGDLSSIRWVCSSLRHAQPTCPGAQLCTVYYASLNFRDIMLATGKLSPDAIPGKWTSQDSLLGMEFSGRDASGKRVMGLVPAKGLATSVLLSPDFLWDVPSNWTLEEAASVPVVYSTAYYALVVRGRVRPGETLLIHSGSGGVGQAAIAIALSLGCRVFTTVGSAEKRAYLQARFPQLDSTSFANSRDTSFEQHVLWHTGGKGVDLVLNSLAEEKLQASVRCLATHGRFLEIGKFDLSQNHPLGMAIFLKNVTFHGVLLDAFFNESSADWREVWALVQAGIRDGVVRPLKCTVFHGAQVEDAFRYMAQGKHIGKVVVQVLAEEPEAVLKGAKPKLMSAISKTFCPAHKSYIIAGGLGGFGLELAQWLIQRGVQKLVLTSRSGIRTGYQAKQVRRWRRQGVQVQVSTSNISSLEGARGLIAEAAQLGPVGGVFNLAVVLRDGLLENQTPEFFQDVCKPKYSGTLNLDRVTREACPELDYFVVFSSVSCGRGNAGQSNYGFANSAMERICEKRRHEGLPGLAVQWGAIGDVGILVETMSTNDTIVSGTLPQRMASCLEVLDLFLNQPHMVLSSFVLAEKAAAYRDRDSQRDLVEAVAHILGIRDLAAVNLDSSLADLGLDSLMSVEVRQTLERELNLVLSVREVRQLTLRKLQELSSKADEASELACPTPKEDGLAQQQTQLNLRSLLVNPEGPTLMRLNSVQSSERPLFLVHPIEGSTTVFHSLASRLSIPTYGLQCTRAAPLDSIHSLAAYYIDCIRQVQPEGPYRVAGYSYGACVAFEMCSQLQAQQSPAPTHNSLFLFDGSPTYVLAYTQSYRAKLTPGCEAEAETEAICFFVQQFTDMEHNRVLEALLPLKGLEERVAAAVDLIIKSHQGLDRQELSFAARSFYYKLRAAEQYTPKAKYHGNVMLLRAKTGGAYGEDLGADYNLSQVCDGKVSVHVIEGDHRTLLEGSGLESIISIIHSSLAEPRVSVREG

SEQ ID NO: 2 - RNA, 8464 nt, Homo sapiens
  source 1..8464: mol_type = mRNA
gagccagagagacggcagcggccccggcctccctctccgccgcgcttcagcctcccgctccgccgcgctccagcctcgctctccgccgcccgcaccgccgcccgcgccctcaccagagcagccatggaggaggtggtgattgccggcatgtccgggaagctgccagagtcggagaacttgcaggagttctgggacaacctcatcggcggtgtggacatggtcacggacgatgaccgtcgctggaaggcggggctctacggcctgccccggcggtccggcaagctgaaggacctgtctaggtttgatgcctccttcttcggagtccaccccaagcaggcacacacgatggaccctcagctgcggctgctgctggaagtcacctatgaagccatcgtggacggaggcatcaacccagattcactccgaggaacacacactggcgtctgggtgggcgtgagcggctctgagacctcggaggccctgagccgagaccccgagacactcgtgggctacagcatggtgggctgccagcgagcgatgatggccaaccggctctccttcttcttcgacttcagagggcccagcatcgcactggacacagcctgctcctccagcctgatggccctgcagaacgcctaccaggccatccacagcgggcagtgccctgccgccatcgtggggggcatcaatgtcctgctgaagcccaacacctccgtgcagttcttgaggctggggatgctcagccccgagggcacctgcaaggccttcgacacagcggggaatgggtactgccgctcggagggtgtggtggccgtcctgctgaccaagaagtccctggcccggcgggtgtacgccaccatcctgaacgccggcaccaatacagatggcttcaaggagcaaggcgtgaccttcccctcaggggatatccaggagcagctcatccgctcgttgtaccagtcggccggagtggcccctgagtcatttgaatacatcgaagcccacggcacaggcaccaaggtgggcgacccccaggagctgaatggcatcacccgagccctgtgcgccacccgccaggagccgctgctcatcggctccaccaagtccaacatggggcacccggagccagcctcggggctggcagccctggccaaggtgctgctgtccctggagcacgggctctgggcccccaacctgcacttccatagccccaaccctgagatcccagcgctgttggatgggcggctgcaggtggtggaccagcccctgcccgtccgtggcggcaacgtgggcatcaactcctttggcttcgggggctccaacgtgcacatcatcctgaggcccaacacgcagccgccccccgcacccgccccacatgccaccctgccccgtctgctgcgggccagcggacgcacccctgaggccgtgcagaagctgctggagcagggcctccggcacagccaggacctggctttcctgagcatgctgaacgacatcgcggctgtccccgccaccgccatgcccttccgtggctacgctgtgctgggtggtgagcgcggtggcccagaggtgcagcaggtgcccgctggcgagcgcccgctctggttcatctgctctgggatgggcacacagtggcgcgggatggggctgagcctcatgcgcctggaccgcttccgagattccatcctacgctccgatgaggctgtgaagccattcggcctgaaggtgtcacagctgctgctgagcacagacgagagcacctttgatgacatcgtccattcgtttgtgagcctgactgccatccagataggcctcatagacctgctgagctgcatggggctgaggccagatggcatcgtcggccactccctgggggaggtggcctgtggctacgccgacggctgcctgtcccaggaggaggccgtcctcgctgcctactggaggggacagtgcatcaaagaagcccatctcccgccgggcgccatggcagccgtgggcttgtcctgggaggagtgtaaacagcgctgccccccgggcgtggtgcccgcctgccacaactccaaggacacagtcaccatctcgggacctcaggccccggtgtttgagttcgtggagcagctgaggaaggagggtgtgtttgccaaggaggtgcggaccggcggtatggccttccactcctacttcatggaggccatcgcacccccactgctgcaggagctcaagaaggtgatccgggagccgaagccacgttcagcccgctggctcagcacctctatccccgaggcccagtggcacagcagcctggcacgcacgtcctccgccgagtacaatgtcaacaacctggtgagccctgtgctgttccaggaggccctgtggcacgtgcctgagcacgcggtggtgctggagatcgcgccccacgccctgctgcaggctgtcctgaagcgtggcctgaagccgagctgcaccatcatccccctgatgaagaaggatcacagggacaacctggagttcttcctggccggcatcggcaggctgcacctctcaggcatcgacgccaaccccaatgccttgttcccacctgtggagttcccagctccccgaggaactcccctcatctccccactcatcaagtgggaccacagcctggcctgggacgtgccggccgccgaggacttccccaacggttcaggttccccctcagccgccatctacaacatcgacaccagctccgagtctcctgaccactacctggtggaccacaccctcgacggtcgcgtcctcttccccgccactggctacctgagcatagtgtggaagacgctggcccgcgccctgggcctgggcgtcgagcagctgcctgtggtgtttgaggatgtggtgctgcaccaggccaccatcctgcccaagactgggacagtgtccctggaggtacggctcctggaggcctcccgtgccttcgaggtgtcagagaacggcaacctggtagtgagtgggaaggtgtaccagtgggatgaccctgaccccaggctcttcgaccacccggaaagccccacccccaaccccacggagcccctcttcctggcccaggctgaagtttacaaggagctgcgtctgcgtggctacgactacggccctcatttccagggcatcctggaggccagcctggaaggtgactcggggaggctgctgtggaaggataactgggtgagcttcatggacaccatgctgcagatgtccatcctgggctcggccaagcacggcctgtacctgcccacccgtgtcaccgccatccacatcgaccctgccacccacaggcagaagctgtacacactgcaggacaaggcccaagtggctgacgtggtggtgagcaggtggctgagggtcacagtggccggaggcgtccacatctccgggctccacactgagtcggccccgcggcggcagcaggagcagcaggtgcccatcctggagaagttttgcttcactccccacacggaggaggggtgcctgtctgagcgcgctgccctgcaggaggagctgcaactgtgcaaggggctggtgcaggcactgcagaccaaggtgacccagcaggggctgaagatggtggtgcccggactggatggggcccagatcccccgggacccctcacagcaggaactgccccggctgttgtcggctgcctgcaggcttcagctcaacgggaacctgcagctggagctggcgcaggtgctggcccaggagaggcccaagctgccagaggaccctctgctcagcggcctcctggactccccggcactcaaggcctgcctggacactgccgtggagaacatgcccagcctgaagatgaaggtggtggaggtgctggctggccacggtcacctgtattcccgcatcccaggcctgctcagcccccatcccctgctgcagctgagctacacggccaccgaccgccacccccaggccctggaggctgcccaggccgagctgcagcagcacgacgttgcccagggccagtgggatcccgcagaccctgcccccagcgccctgggcagcgccgacctcctggtgtgcaactgtgctgtggctgccctcggggacccggcctcagctctcagcaacatggtggctgccctgagagaagggggctttctgctcctgcacacactgctccgggggcaccccctcggggacatcgtggccttcctcacctccactgagccgcagtatggccagggcatcctgagccaggacgcgtgggagagcctcttctccagggtgtcgctgcgcctggtgggcctgaagaagtccttctacggctccacgctcttcctgtgccgccggcccaccccgcaggacagccccatcttcctgccggtggacgataccagcttccgctgggtggagtctctgaagggcatcctggctgacgaagactcttcccggcctgtgtggctgaaggccatcaactgtgccacctcgggcgtggtgggcttggtgaactgtctccgccgagagcccggcgggaaccgcctccggtgtgtgctgctctccaacctcagcagcacctcccacgtcccggaggtggacccgggctccgcagaactgcagaaggtgttgcagggagacctggtgatgaacgtctaccgcgacggggcctggggggctttccgccacttcctgctggaggaggacaagcctgaggagccgacggcacatgcctttgtgagcaccctcacccggggggacctgtcctccatccgctgggtctgctcctcgctgcgccatgcccagcccacctgccctggcgcccagctctgcacggtctactacgcctccctcaacttccgcgacatcatgctggccactggcaagctgtcccctgatgccatcccagggaagtggacctcccaggacagcctgctaggtatggagttctcgggccgagacgccagcggcaagcgtgtgatgggactggtgcctgccaagggcctggccacctctgtcctgctgtcaccggacttcctctgggatgtgccttccaactggacgctggaggaggcggcctcggtgcctgtcgtctacagcacggcctactacgcgctggtggtgcgtgggcgggtgcgccccggggagacgctgctcatccactcgggctcgggcggcgtgggccaggccgccatcgccatcgccctcagtctgggctgccgcgtcttcaccaccgtggggtcggctgagaagcgggcgtacctccaggccaggttcccccagctcgacagcaccagcttcgccaactcccgggacacatccttcgagcagcatgtgctgtggcacacgggcgggaagggcgttgacctggtcttgaactccttggcggaagagaagctgcaggccagcgtgaggtgcttggctacgcacggtcgcttcctggaaattggcaaattcgacctttctcagaaccacccgctcggcatggctatcttcctgaagaacgtgacattccacggggtcctactggatgcgttcttcaacgagagcagtgctgactggcgggaggtgtgggcgcttgtgcaggccggcatccgggatggggtggtacggcccctcaagtgcacggtgttccatggggcccaggtggaggacgccttccgctacatggcccaagggaagcacattggcaaagtcgtcgtgcaggtgcttgcggaggagccggaggcagtgctgaagggggccaaacccaagctgatgtcggccatctccaagaccttctgcccggcccacaagagctacatcatcgctggtggtctgggtggcttcggcctggagttggcgcagtggctgatacagcgtggggtgcagaagctcgtgttgacttctcgctccgggatccggacaggctaccaggccaagcaggtccgccggtggaggcgccagggcgtacaggtgcaggtgtccaccagcaacatcagctcactggagggggcccggggcctcattgccgaggcggcgcagcttgggcccgtgggcggcgtcttcaacctggccgtggtcttgagagatggcttgctggagaaccagaccccagagttcttccaggacgtctgcaagcccaagtacagcggcaccctgaacctggacagggtgacccgagaggcgtgccctgagctggactactttgtggtcttctcctctgtgagctgcgggcgtggcaatgcgggacagagcaactacggctttgccaattccgccatggagcgtatctgtgagaaacgccggcacgaaggcctcccaggcctggccgtgcagtggggcgccatcggcgacgtgggcattttggtggagacgatgagcaccaacgacacgatcgtcagtggcacgctgccccagcgcatggcgtcctgcctggaggtgctggacctcttcctgaaccagccccacatggtcctgagcagctttgtgctggctgagaaggctgcggcctatagggacagggacagccagcgggacctggtggaggccgtggcacacatcctgggcatccgcgacttggctgctgtcaacctggacagctcactggcggacctgggcctggactcgctcatgagcgtggaggtgcgccagacgctggagcgtgagctcaacctggtgctgtccgtgcgcgaggtgcggcaactcacgctccggaaactgcaggagctgtcctcaaaggcggatgaggccagcgagctggcatgccccacgcccaaggaggatggtctggcccagcagcagactcagctgaacctgcgctccctgctggtgaacccggagggccccaccctgatgcggctcaactccgtgcagagctcggagcggcccctgttcctggtgcacccaatcgagggctccaccaccgtgttccacagcctggcctcccggctcagcatccccacctatggcctgcagtgcacccgagctgcgccccttgacagcatccacagcctggctgcctactacatcgactgcatcaggcaggtgcagcccgagggcccctaccgcgtggccggctactcctacggggcctgcgtggcctttgaaatgtgctcccagctgcaggcccagcagagcccagcccccacccacaacagcctcttcctgttcgacggctcgcccacctacgtactggcctacacccagagctaccgggcaaagctgaccccaggctgtgaggctgaggctgagacggaggccatatgcttcttcgtgcagcagttcacggacatggagcacaacagggtgctggaggcgctgctgccgctgaagggcctagaggagcgtgtggcagccgccgtggacctgatcatcaagagccaccagggcctggaccgccaggagctgagctttgcggcccggtccttctactacaagctgcgtgccgctgagcagtacacacccaaggccaagtaccatggcaacgtgatgctactgcgcgccaagacgggtggcgcctacggcgaggacctgggcgcggactacaacctctcccaggtatgcgacgggaaagtatccgtccacgtcatcgagggtgaccaccgcacgctgctggagggcagcggcctggagtccatcatcagcatcatccacagctccctggctgagccacgcgtgagcgtgcgggagggctaggcccgtgcccccgcctgccaccggaggtcactccaccatccccaccccaccccaccccacccccgccatgcaacgggattgaagggtcctgccggtgggaccctgtccggcccagtgccactgccccccgaggctgctagatgtaggtgttaggcatgtcccacccacccgccgcctcccacggcacctcggggacaccagagctgccgacttggagactcctggtctgtgaagagccggtggtgcccgtgcccgcaggaactgggctgggcctcgtgcgcccgtggggtctgcgcttggtctttctgtgcttggatttgcatatttattgcattgctggtagagacccccaggcctgtccaccctgccaagactcctcaggcagcgtgtgggtcccgcactctgcccccatttccccgatgtcccctgcgggcgcgggcagccacccaagcctgctggctgcggccccctctcggccaggcattggctcagcccgctgagtggggggtcgtgggccagtccccgaggagctgggcccctgcacaggcacacagggcccggccacacccagcggccccccgcacagccacccgtggggtgctgcccttatgcccggcgccgggcaccaactccatgtttggtgtttgtctgtgtttgtttttcaagaaatgattcaaattgctgcttggattttgaaatttactgtaactgtcagtgtacacgtctggaccccgtttcatttttacaccaatttggtaaaaatgctgctctcagcctcccacaattaaaccgcatgtgatctcca

SEQ ID NO: 3 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 4 - RNA, 2097 nt, Homo sapiens
  source 1..2097: mol_type = mRNA
gctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaa

SEQ ID NO: 5 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 6 - RNA, 3634 nt, Homo sapiens
  source 1..3634: mol_type = mRNA
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

(19) United States
(12) Patent Application Publication
(10) Pub. No.: US 2026/0053915 A1
(43) Pub. Date: Feb. 26, 2026

(54) COMBINATION OF RADIATION/CHEMOTHERAPY, PD-1 INHIBITOR AND FATTY ACID SYNTHASE INHIBITOR

(71) Applicant: Cornell University, Ithaca, NY (US)

(72) Inventors: Claire Vanpouille-Box et al., New York, NY (US); Mara De Martino, New York, NY (US); Camille Belloche, New York, NY (US); Maria Cecilia Lira, New York, NY (US); Kathryn Beal, New York, NY (US)

(21) Appl. No.: 19/236,662

(22) Filed: Jun. 12, 2025

Related U.S. Application Data
(60) Provisional application No. 63/659,820, filed on Jun. 13, 2024.

(51) Int. Cl. A61K 39/395; A61K 31/09; A61K 31/336; A61K 31/352; A61K 31/353; A61K 31/365; A61K 31/4178; A61K 31/4439; A61K 31/4704; A61K 31/506; A61K 31/519; A61K 39/00; A61K 45/06; A61P 35/00

(52) U.S. Cl. CPC A61K 39/3955; A61K 31/09; A61K 31/336; A61K 31/352; A61K 31/353; A61K 31/365; A61K 31/4178; A61K 31/4439; A61K 31/4704; A61K 31/506; A61K 31/519; A61K 45/06; A61P 35/00; A61K 2039/505

(57) ABSTRACT

Method and compositions for treating glioblastoma (GBM) and other cancers is disclosed, involving the combination of radiation therapy (RT), fatty acid synthase (FASN) inhibitors, and programmed cell death protein 1 (PD-1) inhibitors or programmed death ligand 1 (PD-L1) inhibitors. The disclosed approach addresses the technical problem of RT-induced immunosuppression and resistance in cancers by targeting RT-induced lipid metabolism, which promotes immune escape. FASN inhibitors block fatty acid synthesis, enhancing immune cell infiltration and restoring anti-tumor immunity. PD-1/PD-L1 inhibitors further counteract immunosuppressive pathways to improve therapeutic efficacy. This combination therapy prolongs survival, induces immune memory, and is applicable to other cancers, including those exhibiting increased lipid content post-RT or chemotherapy, including melanoma, breast cancer, and lung cancer.

### PRIORITY

This application claims the benefit of the filing date of U.S. application No. 63/659,820, filed Jun. 13, 2024, the disclosure of which is incorporated by reference herein.

### GOVERNMENT SUPPORT

This invention was made with government support under R01NS131945 awarded by the National Institute of Health. The government has certain rights in the invention.

### INCORPORATION BY REFERENCE OF SEQUENCE LISTING

This application contains a Sequence Listing which has been submitted electronically in ST26 format and hereby incorporated by reference in its entirety. Said ST26 file, created on Jun. 7, 2025, is named 1676213US1.xml and is 23,390 bytes in size.

### BACKGROUND

Glioblastoma (GBM) remains among the most aggressive and challenging forms of brain cancer, with a median survival rate of only 15 months despite advancements in therapeutic approaches such as radiation therapy (RT) and chemotherapy. While RT plays a significant role in GBM treatment, RIT can contribute to immunosuppression and treatment resistance, primarily through mechanisms that are not yet fully understood. Clinical trials utilizing immune checkpoint inhibitors, such as anti-programmed cell death protein 1 (PD-1) therapies, have shown limited efficacy in GBM, with objective response rates as low as 8% in recurrent cases.

### SUMMARY

The present therapeutic approach addresses the treatment limitations by introducing a novel combination therapy that integrates RT, fatty acid synthase (FASN) inhibitors, and PD-1/programmed death ligand 1 (PD-L1) inhibitors to counteract RT-induced immunosuppression. Unlike conventional methods that target GBM through isolated mechanisms, this approach disrupts the tumor's lipid metabolism, which serves as a significant resistance pathway induced by RT. By inhibiting FASN, the therapy prevents the synthesis of fatty acids that shield GBM cells from endoplasmic reticulum (ER) stress and anti-tumor immune response. This metabolic intervention reactivates the cancer cell-mediated type-I interferon (IFN-I) response, thereby facilitating the recruitment and activation of cytotoxic T lymphocytes (CTLs) within the tumor microenvironment. Concurrently, the use of PD-1/PD-L1 inhibitors mitigates immunosuppressive signaling pathways, further supporting the immune system's ability to target and eliminate cancer cells.

The described combination therapy not only improves survival outcomes but also induces durable immune memory, as demonstrated by the ability of treated subjects to resist tumor rechallenge. This approach is broadly applicable to other cancers that exhibit increased lipid content or FASN expression following RT or chemotherapy, including melanoma, breast cancer, and lung cancer. By addressing the root cause of RT-induced resistance and leveraging synergistic interactions between FASN inhibitors and immune checkpoint inhibitors, this development represents a significant advancement in cancer treatment strategies, offering improved therapeutic efficacy and patient survival.

In one embodiment, a method to treat cancer in a subject includes administering an effective amount of a FASN inhibitor together with an effective amount of a PD-1 inhibitor, a PD-L1 inhibitor, or a combination thereof, while the subject is or will be administered RT and/or chemotherapy. In some versions, the inhibitors are administered one or more days prior to RT and/or chemotherapy, with administration continuing concurrently with and optionally following these therapies. In other embodiments, the FASN inhibitor is administered prior to, during, and after RT, while the PD-1 inhibitor and/or PD-L1 inhibitor is administered after RT. This treatment approach will result in prolonged survival relative to treatment without the inhibitors.

In certain embodiments, the RT may be selected from external beam RT, proton therapy, systemic RT, targeted RT, or any combination thereof, and the chemotherapy may be selected from alkylating agents, antimetabolites, mitotic inhibitors, topoisomerase inhibitors, hormonal therapies, immune therapies, or combinations thereof. The PD-1 inhibitor and/or PD-L1 inhibitor may be selected from antibodies, small molecules, inhibitory nucleic acids, or combinations thereof, with specific embodiments including PD-1 inhibitors such as pembrolizumab, nivolumab, cemiplimab, tislelizumab, dostarlimab, retifanlimab, toripalimab or combinations thereof, and PD-L1 inhibitors such as atezolizumab, avelumab, cosibelimab, durvalumab or combinations thereof. Likewise, the FASN inhibitor may be selected from antibodies, small molecules, inhibitory nucleic acids, or combinations thereof, with examples including cerulenin, C75, orlistat, Fasnall, TVB-2640, TVB-3664, TVB-3567, triclosan, GSK837149A, bisamide scaffold, hydroxyquinoline-2(1H)-one scaffold, platensimycin, epigallocatechin gallate (EGCG) or combinations thereof. In some embodiments, the treatment is particularly applicable to cancers that exhibit an increased lipid content after radiation or chemotherapy, and the cancer may be selected from melanoma, prostate cancer, breast cancer, bladder cancer, lung cancer, neuroglioma, diffuse midline glioma, or combinations thereof.

In another embodiment, a kit for treating cancer is provided that comprises a first pharmaceutical composition including a PD-1 inhibitor and/or a PD-L1 inhibitor, a second pharmaceutical composition including a FASN inhibitor, and instructions for administering RT and/or chemotherapy in conjunction with the pharmaceutical compositions.

### DRAWINGS

The drawings illustrate generally, by way of example, but not by way of limitation, various embodiments discussed herein.

FIGS. 1A-IC. FASN inhibits RT-mediated nucleic acid sensing and IFN-I production. CT2A derivatives were induced with doxycycline for 10 days and irradiated with a single dose of 10 Gy. 24 hrs post RT, supernatants and cells were collected to quantify IFN-beta and CXCL 10 (A). CGAS, RIG-I or MDA5 were knock-down (KD) in CT2A derivatives using siRNA technology. Cells were then induced for 10 days with doxycycline and subjected to a single dose of 10 Gy. 24 hrs post-RT, protein analysis and quantification of IFN-beta was performed. (B) Validation of CGAS, RIG-1 and MDA5 KD. (C) IFN-beta secretion in CT2A derivatives carrying siRNA against cGAS, RIG-I or MDA5. n=3/condition; One-way ANOVA, post-hoc Tukey's comparison. Duplicate experiment.

FIGS. 2A-2C. Genetic FASN blockade promotes the recruitment of immune cells in irradiated GBM. (A) experiment scheme. CT2A murine GBM cells unable to express FASN (CT2AshFASN) or its non-silencing control (CT2AshNS) were injected intracranially on day 0. Starting day 6 post cell injection, mice were fed with doxycycline to remove FASN expression in vivo (B). On day 10, mice received focal irradiation selectively to the intracranial tumor (10 Gy). On day 18, brains were collected to assess immune cells infiltration using multiplex immunofluorescence (C).

FIG. 3. Pharmacologic FASN blockade promote the recruitment of immune cells in irradiated GBM. GL261 murine GBM cells were injected intracranially on day 0. Starting day 9, mice received i.p. injections of a pharmacologic FASN inhibitor (5 mg/kg) daily until day 30. Radiation was administered on day 10 (10 Gy) selectively to the intracranial tumor. On day 30, mouse brains were collected to assess the infiltration of CD8+ and CD11c+ cells infiltrating GBM by immunofluorescence. n=3-4 mice per group. T-test.

FIG. 4. FASN knockdown facilitates IFN-I dependent immune cells infiltrates in GBM. Syngeneic C57Bl/6 animals received intracerebral injections of CT2A derivatives in day 0. On day 6, animals were fed with doxycycline to induce FASN knockdown. On day 10, focal irradiation was performed. Animals received anti-IFNAR monoclonal antibody i.p. (200 ug/mouse) on days 6, 10 and 14. On day 17, brains were and flash frozen to stain for CD8 (green). n=3/conditions; One large symbol represents the mean of at least 3 different pictures (small symbols) of 1 animal. duplicate experiment. One-way ANOVA, Tukey post hoc modification

FIG. 5. Irradiation of GBM cells promotes the release of prostaglandin E2 (PGE2). Murine GBM cells were subjected to 10 Gy irradiation in vitro. Twenty-four hours post RT, supernatant was collected to quantify PGE2 by ELISA. n=3/group. T-test.

FIG. 6. Blockade of FASN reduces the infiltration of terminally exhausted T cells in mice treated with RT and anti-PD-1. GL261 cells were injected i.e. into C57BL/6N mice on day 0 (n=4-5/group). Starting day 10, 5 daily fractions of 6 Gy focal irradiation were performed using the SARRP irradiator equipped with a CT scan. FASNi (Fasnall) was administered i.p. between days 9-30 (5 mg/kg). Anti-PD-1 antibody was administered i.p. between days 15-20. On day 20, tumor-bearing brains were collected and digested to perform flow cytometry analysis. One-way ANOVA, Tukey post-test, *p<0.001.

FIGS. 7A-7C. FASNi in combination with focal radiation therapy and anti-PD-1 prolongs survival of GBM bearing mice. A. Experiment scheme and timeline: GL261 cells were injected i.e. into C57BL/6N mice on day 0 (n=7/group). Starting day 10, 5 daily fractions of 6 Gy focal irradiation were performed using the SARRP irradiator equipped with a CT scan. FASNi (Fasnall) was administered i.p. between days 9-30. Anti-PD-1 antibody was administered i.p. between days 15-24. Survival was monitored for 100 days, and Kaplan Meyer survival curve and median survival values are reported (B). Mice that remained tumor free for at least 100 days were rechallenged with GL261 cells injection in the contralateral hemisphere and followed up for survival.

FIG. 8. Accumulation of lipid droplet post RT is not unique to GBM.

FIG. 9. Chemotherapies also promote the accumulation of lipids.

FIG. 10. Pan-cancer Correlation between FASN exp with Hallmark pathways.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Glioblastoma is a devastating disease with a dismal prognosis. No improvement of overall survival has been made over the last two decades. Previous clinical trials using anti-PD-1 alone led to only an 8% objective response rate in recurrent GBM (Checkmate 143) (Reardon et al., JAMA Oncol 2020). As all patients from Checkmate 143 received prior RT and temozolomide (TMZ), it is conceivable that RT-induced fatty acid synthase (FASN) may prevent infiltration of T cells into GBM and promote PD-1/PD-L1 immunosuppression, thus explaining the lack of response to anti-PD-1 in these patients. Provided herein is a unique design to block RT-induced lipid metabolism and restore immune reactivity against GBM by inhibiting FASN. This triple combination (i.e. RT+FASNi+anti-PD1) is designed not only to prevent RT-induced resistance but also to trigger long-lasting anti-tumor immunity against GBM.

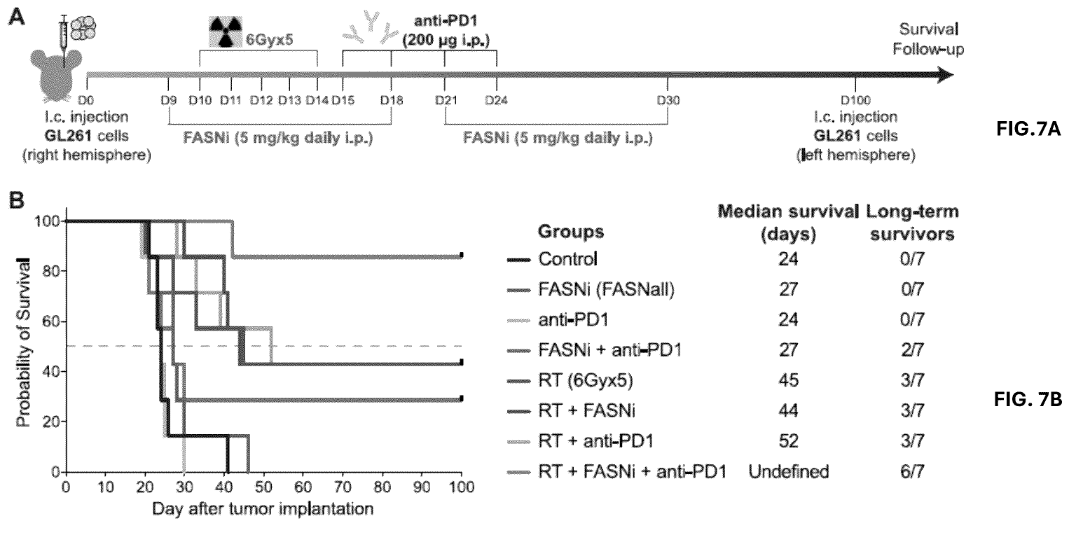

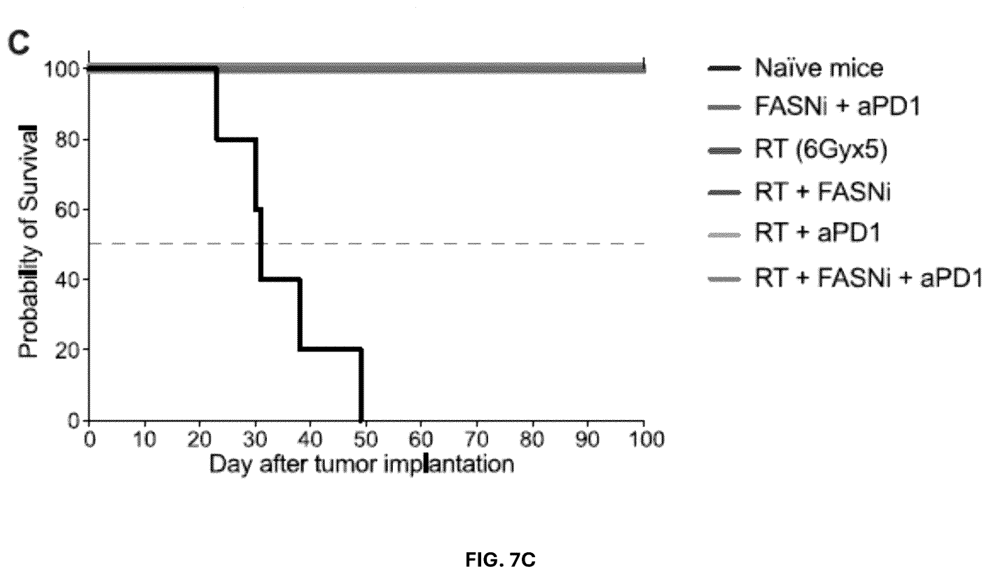

In one embodiment, a combination strategy to control the tumor and to induce protective immune memory is depicted in FIG. 7A:
Daily administration of FASNi (5 mg/kg; i.p.) starting a day prior irradiation (i.e. day 9) for 10 days (until day 18). After a 2 days break, mice received FASNi (5 mg/kg; i.p.) for an additional period of 10 days (from day 21 to day 30);
Irradiation: starting day 10 post tumor cells injection, mice received daily irradiation of 6 Gy for 5 days (5×6 Gy).
From day 15 to day 24; anti-PD-1 was administered i.p. twice a week (i.e. day 15, day 18, day 21 and day 24).

The sequencing of drugs and the radiation schedule of this combination can be modified.

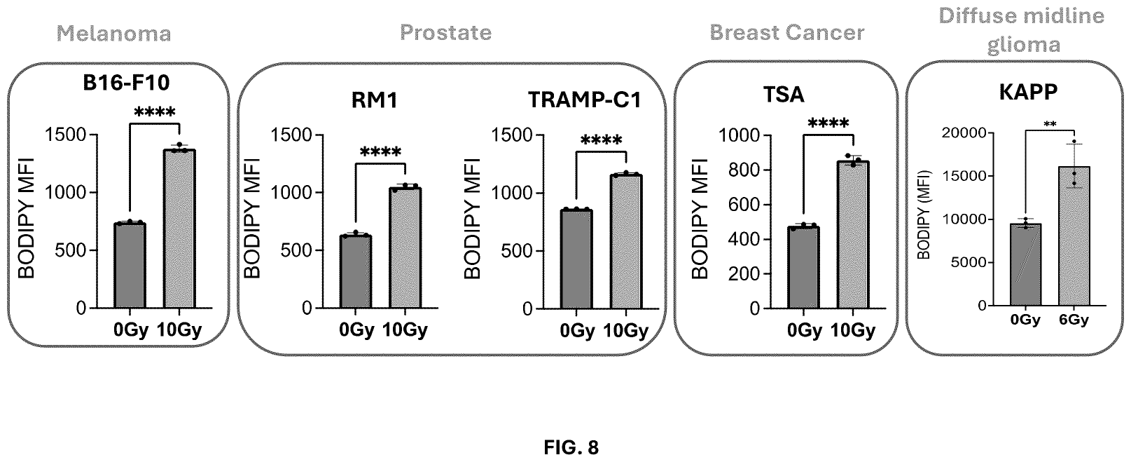

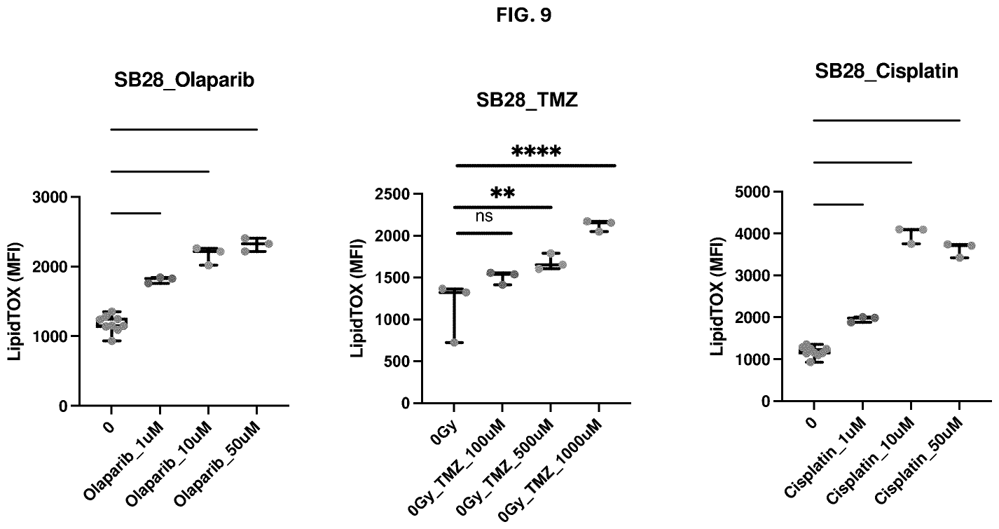

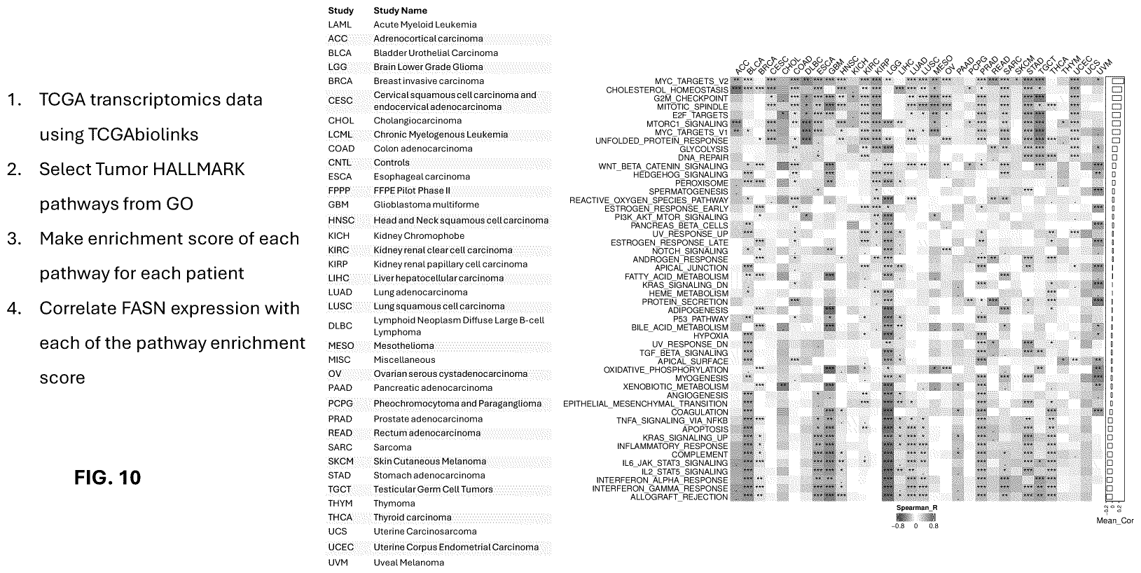

In one embodiment, this combination therapy is used in the treatment/management of GBM. It can also be used with other irradiated tumors that increase their lipid content post irradiation as a resistance mechanism, such as melanoma, prostate cancer, breast cancer, bladder cancer, lung cancer, glioma and diffuse midline glioma (FIG. 8) or post chemotherapy, such as breast cancer (FIG. 9) or FASN expression (FIG. 10).

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of 5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

“Homologous” or “identity” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “comparing” refers to making an assessment of how the proportion, level or cellular localization of one or more biomarkers in a sample from a patient relates to the proportion, level or cellular localization of the corresponding one or more biomarkers in a standard or control sample. For example, “comparing” may refer to assessing whether the proportion, level, or cellular localization of one or more biomarkers in a sample from a patient is the same as, more or less than, or different from the proportion, level, or cellular localization of the corresponding one or more biomarkers in a standard or control sample or another patient sample. More specifically, the term may refer to assessing whether the proportion, level, or cellular localization of one or more biomarkers in a sample from a patient is the same as, more or less than, different from or otherwise corresponds (or not) to the proportion, level, or cellular localization of predefined biomarker levels that correspond to, for example, a cancer patient.

As used herein, the terms “indicates” or “correlates” (or “indicating” or “correlating,” or “indication” or “correlation,” depending on the context) in reference to a parameter, e.g., a modulated proportion, level, or cellular localization in a sample from a patient, may mean that the patient has or at risk of having cancer.

The terms “patient,” “individual,” or “subject” are used interchangeably herein, and refer to a mammal, particularly, a human. As used herein, a “subject in need thereof” is a patient, animal, mammal, or human, who will benefit from the method of this invention. The patient may have mild, intermediate or severe disease, or may only experience transient, or fluctuating symptoms. The patient may be an individual, at risk of developing a disease, in need of treatment or in need of diagnosis based on particular symptoms or family history. In some cases, the terms may refer to treatment of animals, in such as veterinary applications (e.g., companion animals (e.g., cats or dogs) or livestock (e.g., pigs, cows, horses)).

The terms “measuring” and “determining” are used interchangeably throughout and refer to methods which include obtaining a patient sample and/or detecting the level of a biomarker(s) in a sample. In one embodiment, the terms refer to obtaining a patient sample and detecting the level of one or more biomarkers in the sample. In another embodiment, the terms “measuring” and “determining” mean detecting level of one or more biomarkers in a patient sample. Measuring can be accomplished by methods known in the art and those further described herein including. The term “measuring” is also used interchangeably throughout with the term “detecting.”

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms. Moreover, a sample obtained from a patient can be divided and only a portion may be used for diagnosis. Further, the sample, or a portion thereof, can be stored under conditions to maintain sample for later analysis.

Various methodologies of the instant invention include a step that involves comparing a value, level, feature, characteristic, property, etc. to a “suitable control,” referred to interchangeably herein as an “appropriate control” or a “control sample.” A “suitable control,” “appropriate control” or a “control sample” is any control or standard familiar to one of ordinary skill in the art useful for comparison purposes. In one embodiment, a “suitable control” or “appropriate control” is a value, level, feature, characteristic, property, etc., determined in a cell, organ, or patient, e.g., a control or normal cell, organ, or patient, exhibiting, for example, normal traits. For example, the biomarkers of the present invention may be assayed in a sample from an unaffected individual (UI) or a normal control individual (NC) (both terms are used interchangeably herein) or other affected individual. In another embodiment, a “suitable control” or “appropriate control” is a value, level, feature, characteristic, property, etc. determined prior to performing a therapy on a patient. In yet another embodiment, a transcription rate, mRNA level, translation rate, protein level, biological activity, cellular characteristic or property, genotype, phenotype, etc. can be determined prior to, during, or after administering a therapy into a cell, organ, or patient. In a further embodiment, a “suitable control” or “appropriate control” is a predefined value, level, feature, characteristic, property, etc.

The term “otherwise identical sample,” as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating,” “treat” and “treatment” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

A disease, condition, or disorder is “alleviated” if the severity of a symptom of the disease or disorder, the frequency with which such a symptom is experienced by a subject, or both, are reduced.

As used herein “injecting, administering or applying” includes administration of the invention by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

As used herein, the term “pharmaceutically acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

As used herein, an “instructional material” includes a publication, a recording, a diagram, or any other medium of expression which can be used to communicate the usefulness of the of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified invention or be shipped together with a container. Alternatively, the instructional material may be shipped separately from the container with the intention that the instructional material and the invention be used cooperatively by the recipient.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes,” “including” and the like. As used herein, “including” or “includes” or the like means including, without limitation.

Methods involving conventional molecular biology techniques are described herein. Such techniques are generally known in the art and are described in detail in methodology treatises, such as Molecular Cloning: A Laboratory Manual, Sambrook et al., Cold Spring Harbor Laboratory Press, Cold Spring Harbor, N.Y.

### FASN Inhibitors

Fatty acid synthase (FASN) is an enzyme (E number 2.3.1.85) that in humans is encoded by the FASN gene. FASN is a multi-enzyme protein that catalyzes fatty acid synthesis. It is not a single enzyme, but an enzymatic system composed of two identical 272 kDa multifunctional polypeptides (A. Jayakumar et al. Proc Natl Acad Sci USA. 1995 Sep. 12; 92(19):8695-8699), in which substrates are handed from one functional domain to the next. Its main function is to catalyze the synthesis of palmitate (C16:0, a long-chain saturated fatty acid) from acetyl-CoA and malonyl-CoA, in the presence of NADPH.

Exemplary sequence of human FASN protein can be found at accession number NP_004095, the sequence is provided below:

| (SEQ ID NO: 1) | |
|---|---|
| 1 | MEEVVIAGMS GKLPESENLQ EFWDNLIGGV DMVTDDDRRW KAGLYGLPRR SGKLKDLSRF |
| 61 | DASFFGVHPK QAHTMDPQLR LLLEVTYEAI VDGGINPDSL RGTHTGVWVG VSGSETSEAL |
| 121 | SRDPETLVGY SMVGCQRAMM ANRLSFFFDF RGPSIALDTA CSSSLMALQN AYQAIHSGQC |
| 181 | PAAIVGGINV LLKPNTSVQF LRLGMLSPEG TCKAFDTAGN GYCRSEGVVA VLLTKKSLAR |
| 241 | RVYATILNAG TNTDGFKEQG VTFPSGDIQE QLIRSLYQSA GVAPESFEYI EAHGTGTKVG |
| 301 | DPQELNGITR ALCATRQEPL LIGSTKSNMG HPEPASGLAA LAKVLLSLEH GLWAPNLHFH |
| 361 | SPNPEIPALL DGRLQVVDQP LPVRGGNVGI NSFGFGGSNV HIILRPNTQP PPAPAPHATL |
| 421 | PRLLRASGRT PEAVQKLLEQ GLRHSQDLAF LSMLNDIAAV PATAMPFRGY AVLGGERGGP |
| 481 | EVQQVPAGER PLWFICSGMG TQWRGMGLSL MRLDRFRDSI LRSDEAVKPF GLKVSQLLLS |
| 541 | TDESTFDDIV HSFVSLTAIQ IGLIDLLSCM GLRPDGIVGH SLGEVACGYA DGCLSQEEAV |
| 601 | LAAYWRGQCI KEAHLPPGAM AAVGLSWEEC KQRCPPGVVP ACHNSKDTVT ISGPQAPVFE |
| 661 | FVEQLRKEGV FAKEVRTGGM AFHSYFMEAI APPLLQELKK VIREPKPRSA RWLSTSIPEA |
| 721 | QWHSSLARTS SAEYNVNNLV SPVLFQEALW HVPEHAVVLE IAPHALLQAV LKRGLKPSCT |
| 781 | IIPLMKKDHR DNLEFFLAGI GRLHLSGIDA NPNALFPPVE FPAPRGTPLI SPLIKWDHSL |
| 841 | AWDVPAAEDF PNGSGSPSAA IYNIDTSSES PDHYLVDHTL DGRVLFPATG YLSIVWKTLA |
| 901 | RALGLGVEQL PVVFEDVVLH QATILPKTGT VSLEVRLLEA SRAFEVSENG NLVVSGKVYQ |
| 961 | WDDPDPRLFD HPESPTPNPT EPLFLAQAEV YKELRLRGYD YGPHFQGILE ASLEGDSGRL |
| 1021 | LWKDNWVSFM DTMLQMSILG SAKHGLYLPT RVTAIHIDPA THRQKLYTLQ DKAQVADVVV |
| 1081 | SRWLRVTVAG GVHISGLHTE SAPRRQQEQQ VPILEKFCFT PHTEEGCLSE RAALQEELQL |
| 1141 | CKGLVQALQT KVTQQGLKMV VPGLDGAQIP RDPSQQELPR LLSAACRLQL NGNLQLELAQ |
| 1201 | VLAQERPKLP EDPLLSGLLD SPALKACLDT AVENMPSLKM KVVEVLAGHG HLYSRIPGLL |
| 1261 | SPHPLLQLSY TATDRHPQAL EAAQAELQQH DVAQGQWDPA DPAPSALGSA DLLVCNCAVA |
| 1321 | ALGDPASALS NMVAALREGG FLLLHTLLRG HPLGDIVAFL TSTEPQYGQG ILSQDAWESL |
| 1381 | FSRVSLRLVG LKKSFYGSTL FLCRRPTPQD SPIFLPVDDT SFRWVESLKG ILADEDSSRP |
| 1441 | VWLKAINCAT SGVVGLVNCL RREPGGNRLR CVLLSNLSST SHVPEVDPGS AELQKVLQGD |
| 1501 | LVMNVYRDGA WGAFRHFLLE EDKPEEPTAH AFVSTLTRGD LSSIRWVCSS LRHAQPTCPG |
| 1561 | AQLCTVYYAS LNFRDIMLAT GKLSPDAIPG KWTSQDSLLG MEFSGRDASG KRVMGLVPAK |
| 1621 | GLATSVLLSP DFLWDVPSNW TLEEAASVPV VYSTAYYALV VRGRVRPGET LLIHSGSGGV |
| 1681 | GQAAIAIALS LGCRVFTTVG SAEKRAYLQA RFPQLDSTSF ANSRDTSFEQ HVLWHTGGKG |
| 1741 | VDLVLNSLAE EKLQASVRCL ATHGRFLEIG KFDLSQNHPL GMAIFLKNVT FHGVLLDAFF |
| 1801 | NESSADWREV WALVQAGIRD GVVRPLKCTV FHGAQVEDAF RYMAQGKHIG KVVVQVLAEE |
| 1861 | PEAVLKGAKP KLMSAISKTF CPAHKSYIIA GGLGGFGLEL AQWLIQRGVQ KLVLTSRSGI |
| 1921 | RTGYQAKQVR RWRRQGVQVQ VSTSNISSLE GARGLIAEAA QLGPVGGVFN LAVVLRDGLL |
| 1981 | ENQTPEFFQD VCKPKYSGTL NLDRVTREAC PELDYFVVFS SVSCGRGNAG QSNYGFANSA |
| 2041 | MERICEKRRH EGLPGLAVQW GAIGDVGILV ETMSTNDTIV SGTLPQRMAS CLEVLDLFLN |
| 2101 | QPHMVLSSFV LAEKAAAYRD RDSQRDLVEA VAHILGIRDL AAVNLDSSLA DIGLDSLMSV |
| 2161 | EVRQTLEREL NLVLSVREVR QLTLRKLQEL SSKADEASEL ACPTPKEDGL AQQQTQLNLR |
| 2221 | SLLVNPEGPT LMRLNSVQSS ERPLFLVHPI EGSTTVFHSL ASRLSIPTYG LQCTRAAPLD |
| 2281 | SIHSLAAYYI DCIRQVQPEG PYRVAGYSYG ACVAFEMCSQ LQAQQSPAPT HNSLFLFDGS |
| 2341 | PTYVLAYTQS YRAKLTPGCE AEAETEAICF FVQQFTDMEH NRVLEALLPL KGLEERVAAA |
| 2401 | VDLIIKSHQG LDRQELSFAA RSFYYKLRAA EQYTPKAKYH GNVMLLRAKT GGAYGEDLGA |
| 2461 | DYNLSQVCDG KVSVHVIEGD HRTLLEGSGL ESIISIIHSS LAEPRVSVRE G, |

or a sequence with at least 55%, 60%, 70%, 80%, 90%, 95%, 96%, 97%, 98%, 99% sequence identity thereto. Identity/alignment may be ascertained or conducted using, for example, the homology alignment algorithm of Needleman and Wunsch, J. Mol. Biol. 48:443-53 (1970).

The human FASN protein can be coded for by the following mRNA (accession number NM 004104.5):

| (SEQ ID NO: 2) | |
|---|---|
| 1 | gagccagaga gacggcagcg gccccggcct ccctctccgc cgcgcttcag cctcccgctc |
| 61 | cgccgcgctc cagcctcgct ctccgccgcc cgcaccgccg cccgcgccct caccagagca |
| 121 | gccatggagg aggtggtgat tgccggcatg tccgggaagc tgccagagtc ggagaacttg |
| 181 | caggagttct gggacaacct catcggcggt gtggacatgg tcacggacga tgaccgtcgc |
| 241 | tggaaggcgg ggctctacgg cctgccccgg cggtccggca agctgaagga cctgtctagg |
| 301 | tttgatgcct ccttcttcgg agtccacccc aagcaggcac acacgatgga ccctcagctg |
| 361 | cggctgctgc tggaagtcac ctatgaagcc atcgtggacg gaggcatcaa cccagattca |
| 421 | ctccgaggaa cacacactgg cgtctgggtg ggcgtgagcg gctctgagac ctcggaggcc |
| 481 | ctgagccgag accccgagac actcgtgggc tacagcatgg tgggctgcca gcgagcgatg |
| 541 | atggccaacc ggctctcctt cttcttcgac ttcagagggc ccagcatcgc actggacaca |
| 601 | gcctgctcct ccagcctgat ggccctgcag aacgcctacc aggccatcca cagcgggcag |
| 661 | tgccctgccg ccatcgtggg gggcatcaat gtcctgctga agcccaacac ctccgtgcag |
| 721 | ttcttgaggc tggggatgct cagccccgag ggcacctgca aggccttcga cacagcgggg |
| 781 | aatgggtact gccgctcgga gggtgtggtg gccgtcctgc tgaccaagaa gtccctggcc |
| 841 | cggcgggtgt acgccaccat cctgaacgcc ggcaccaata cagatggctt caaggagcaa |
| 901 | ggcgtgacct tcccctcagg ggatatccag gagcagctca tccgctcgtt gtaccagtcg |
| 961 | gccggagtgg cccctgagtc atttgaatac atcgaagccc acggcacagg caccaaggtg |
| 1021 | ggcgaccccc aggagctgaa tggcatcacc cgagccctgt gcgccacccg ccaggagccg |
| 1081 | ctgctcatcg gctccaccaa gtccaacatg gggcacccgg agccagcctc ggggctggca |
| 1141 | gccctggcca aggtgctgct gtccctggag cacgggctct gggcccccaa cctgcacttc |
| 1201 | catagcccca accctgagat cccagcgctg ttggatgggc ggctgcaggt ggtggaccag |
| 1261 | cccctgcccg tccgtggcgg caacgtgggc atcaactcct ttggcttcgg gggctccaac |
| 1321 | gtgcacatca tcctgaggcc caacacgcag ccgccccccg cacccgcccc acatgccacc |
| 1381 | ctgccccgtc tgctgcgggc cagcggacgc acccctgagg ccgtgcagaa gctgctggag |
| 1441 | cagggcctcc ggcacagcca ggacctggct ttcctgagca tgctgaacga catcgcggct |
| 1501 | gtccccgcca ccgccatgcc cttccgtggc tacgctgtgc tgggtggtga gcgcggtggc |
| 1561 | ccagaggtgc agcaggtgcc cgctggcgag cgcccgctct ggttcatctg ctctgggatg |
| 1621 | ggcacacagt ggcgcgggat ggggctgagc ctcatgcgcc tggaccgctt ccgagattcc |
| 1681 | atcctacgct ccgatgaggc tgtgaagcca ttcggcctga aggtgtcaca gctgctgctg |
| 1741 | agcacagacg agagcacctt tgatgacatc gtccattcgt ttgtgagcct gactgccatc |
| 1801 | cagataggcc tcatagacct gctgagctgc atggggctga ggccagatgg catcgtcggc |
| 1861 | cactccctgg gggaggtggc ctgtggctac gccgacggct gcctgtccca ggaggaggcc |
| 1921 | gtcctcgctg cctactggag gggacagtgc atcaaagaag cccatctccc gccgggcgcc |
| 1981 | atggcagccg tgggcttgtc ctgggaggag tgtaaacagc gctgcccccc gggcgtggtg |
| 2041 | cccgcctgcc acaactccaa ggacacagtc accatctcgg gacctcaggc cccggtgttt |
| 2101 | gagttcgtgg agcagctgag gaaggagggt gtgtttgcca aggaggtgcg gaccggcggt |
| 2161 | atggccttcc actcctactt catggaggcc atcgcacccc cactgctgca ggagctcaag |
| 2221 | aaggtgatcc gggagccgaa gccacgttca gcccgctggc tcagcacctc tatccccgag |
| 2281 | gcccagtggc acagcagcct ggcacgcacg tcctccgccg agtacaatgt caacaacctg |
| 2341 | gtgagccctg tgctgttcca ggaggccctg tggcacgtgc ctgagcacgc ggtggtgctg |
| 2401 | gagatcgcgc cccacgccct gctgcaggct gtcctgaagc gtggcctgaa gccgagctgc |
| 2461 | accatcatcc ccctgatgaa gaaggatcac agggacaacc tggagttctt cctggccggc |
| 2521 | atcggcaggc tgcacctctc aggcatcgac gccaacccca atgccttgtt cccacctgtg |
| 2581 | gagttcccag ctccccgagg aactcccctc atctccccac tcatcaagtg ggaccacagc |
| 2641 | ctggcctggg acgtgccggc cgccgaggac ttccccaacg gttcaggttc cccctcagcc |
| 2701 | gccatctaca acatcgacac cagctccgag tctcctgacc actacctggt ggaccacacc |
| 2761 | ctcgacggtc gcgtcctctt ccccgccact ggctacctga gcatagtgtg gaagacgctg |
| 2821 | gcccgcgccc tgggcctggg cgtcgagcag ctgcctgtgg tgtttgagga tgtggtgctg |
| 2881 | caccaggcca ccatcctgcc caagactggg acagtgtccc tggaggtacg gctcctggag |
| 2941 | gcctcccgtg ccttcgaggt gtcagagaac ggcaacctgg tagtgagtgg gaaggtgtac |
| 3001 | cagtgggatg accctgaccc caggctcttc gaccacccgg aaagccccac ccccaacccc |
| 3061 | acggagcccc tcttcctggc ccaggctgaa gtttacaagg agctgcgtct gcgtggctac |
| 3121 | gactacggcc ctcatttcca gggcatcctg gaggccagcc tggaaggtga ctcggggagg |
| 3181 | ctgctgtgga aggataactg ggtgagcttc atggacacca tgctgcagat gtccatcctg |
| 3241 | ggctcggcca agcacggcct gtacctgccc acccgtgtca ccgccatcca catcgaccct |
| 3301 | gccacccaca ggcagaagct gtacacactg caggacaagg cccaagtggc tgacgtggtg |
| 3361 | gtgagcaggt ggctgagggt cacagtggcc ggaggcgtcc acatctccgg gctccacact |
| 3421 | gagtcggccc cgcggcggca gcaggagcag caggtgccca tcctggagaa gttttgcttc |
| 3481 | actccccaca cggaggaggg gtgcctgtct gagcgcgctg ccctgcagga ggagctgcaa |
| 3541 | ctgtgcaagg ggctggtgca ggcactgcag accaaggtga cccagcaggg gctgaagatg |
| 3601 | gtggtgcccg gactggatgg ggcccagatc ccccgggacc cctcacagca ggaactgccc |
| 3661 | cggctgttgt cggctgcctg caggcttcag ctcaacggga acctgcagct ggagctggcg |
| 3721 | caggtgctgg cccaggagag gcccaagctg ccagaggacc ctctgctcag cggcctcctg |
| 3781 | gactccccgg cactcaaggc ctgcctggac actgccgtgg agaacatgcc cagcctgaag |
| 3841 | atgaaggtgg tggaggtgct ggctggccac ggtcacctgt attcccgcat cccaggcctg |
| 3901 | ctcagccccc atcccctgct gcagctgagc tacacggcca ccgaccgcca cccccaggcc |
| 3961 | ctggaggctg cccaggccga gctgcagcag cacgacgttg cccagggcca gtgggatccc |
| 4021 | gcagaccctg cccccagcgc cctgggcagc gccgacctcc tggtgtgcaa ctgtgctgtg |
| 4081 | gctgccctcg gggacccggc ctcagctctc agcaacatgg tggctgccct gagagaaggg |
| 4141 | ggctttctgc tcctgcacac actgctccgg gggcaccccc tcggggacat cgtggccttc |
| 4201 | ctcacctcca ctgagccgca gtatggccag ggcatcctga gccaggacgc gtgggagagc |
| 4261 | ctcttctcca gggtgtcgct gcgcctggtg ggcctgaaga agtccttcta cggctccacg |
| 4321 | ctcttcctgt gccgccggcc caccccgcag gacagcccca tcttcctgcc ggtggacgat |
| 4381 | accagcttcc gctaggtgga gtctctgaag ggcatcctgg ctgacgaaga ctcttcccgg |
| 4441 | cctgtgtggc tgaaggccat caactgtgcc acctcgggcg tggtgggctt ggtgaactgt |
| 4501 | ctccgccgag agcccggcgg gaaccgcctc cggtgtgtgc tgctctccaa cctcagcagc |
| 4561 | acctcccacg tcccggaggt ggacccgggc tccgcagaac tgcagaaggt gttgcaggga |
| 4621 | gacctggtga tgaacgtcta ccgcgacggg gcctgggggg ctttccgcca cttcctgctg |
| 4681 | gaggaggaca agcctgagga gccgacggca catgcctttg tgagcaccct cacccggggg |
| 4741 | gacctgtcct ccatccgctg ggtctgctcc tcgctgcgcc atgcccagcc cacctgccct |
| 4801 | ggcgcccagc tctgcacggt ctactacgcc tccctcaact tccgcgacat catgctggcc |
| 4861 | actggcaagc tgtcccctga tgccatccca gggaagtgga cctcccagga cagcctgcta |
| 4921 | ggtatggagt tctcgggccg agacgccagc ggcaagcgtg tgatgggact ggtgcctgcc |
| 4981 | aagggcctgg ccacctctgt cctgctgtca ccggacttcc tctgggatgt gccttccaac |
| 5041 | tggacgctgg aggaggcggc ctcggtgcct gtcgtctaca gcacggccta ctacgcgctg |
| 5101 | gtggtgcgtg ggcgggtgcg ccccggggag acgctgctca tccactcggg ctcgggcggc |
| 5161 | gtgggccagg ccgccatcgc catcgccctc agtctgggct gccgcgtctt caccaccgtg |
| 5221 | gggtcggctg agaagcgggc gtacctccag gccaggttcc cccagctcga cagcaccagc |
| 5281 | ttcgccaact cccgggacac atccttcgag cagcatgtgc tgtggcacac gggcgggaag |
| 5341 | ggcgttgacc tggtcttgaa ctccttggcg gaagagaagc tgcaggccag cgtgaggtgc |
| 5401 | ttggctacgc acggtcgctt cctggaaatt ggcaaattcg acctttctca gaaccacccg |
| 5461 | ctcggcatgg ctatcttcct gaagaacgtg acattccacg gggtcctact ggatgcgttc |
| 5521 | ttcaacgaga gcagtgctga ctggcgggag gtgtgggcgc ttgtgcaggc cggcatccgg |
| 5581 | gatggggtgg tacggcccct caagtgcacg gtgttccatg gggcccaggt ggaggacgcc |
| 5641 | ttccgctaca tggcccaagg gaagcacatt ggcaaagtcg tcgtgcaggt gcttgcggag |
| 5701 | gagccggagg cagtgctgaa gggggccaaa cccaagctga tgtcggccat ctccaagacc |
| 5761 | ttctgcccgg cccacaagag ctacatcatc gctggtggtc tgggtggctt cggcctggag |
| 5821 | ttggcgcagt ggctgataca gcgtggggtg cagaagctcg tgttgacttc tcgctccggg |
| 5881 | atccggacag gctaccaggc caagcaggtc cgccggtgga ggcgccaggg cgtacaggtg |
| 5941 | caggtgtcca ccagcaacat cagctcactg gagggggccc ggggcctcat tgccgaggcg |
| 6001 | gcgcagcttg ggcccgtggg cggcgtcttc aacctggccg tggtcttgag agatggcttg |
| 6061 | ctggagaacc agaccccaga gttcttccag gacgtctgca agcccaagta cagcggcacc |
| 6121 | ctgaacctgg acagggtgac ccgagaggcg tgccctgagc tggactactt tgtggtcttc |
| 6181 | tcctctgtga gctgcgggcg tggcaatgcg ggacagagca actacggctt tgccaattcc |
| 6241 | gccatggagc gtatctgtga gaaacgccgg cacgaaggcc tcccaggcct ggccgtgcag |
| 6301 | tggggcgcca tcggcgacgt gggcattttg gtggagacga tgagcaccaa cgacacgatc |
| 6361 | gtcagtggca cgctgcccca gcgcatggcg tcctgcctgg aggtgctgga cctcttcctg |
| 6421 | aaccagcccc acatggtcct gagcagcttt gtgctggctg agaaggctgc ggcctatagg |
| 6481 | gacagggaca gccagcggga cctggtggag gccgtggcac acatcctggg catccgcgac |
| 6541 | ttggctgctg tcaacctgga cagctcactg gcggacctgg gcctggactc gctcatgagc |
| 6601 | gtggaggtgc gccagacgct ggagcgtgag ctcaacctgg tgctgtccgt gcgcgaggtg |
| 6661 | cggcaactca cgctccggaa actgcaggag ctgtcctcaa aggcggatga ggccagcgag |
| 6721 | ctggcatgcc ccacgcccaa ggaggatggt ctggcccagc agcagactca gctgaacctg |
| 6781 | cgctccctgc tggtgaaccc ggagggcccc accctgatgc ggctcaactc cgtgcagagc |
| 6841 | tcggagcggc ccctgttcct ggtgcaccca atcgagggct ccaccaccgt gttccacagc |
| 6901 | ctggcctccc ggctcagcat ccccacctat ggcctgcagt gcacccgagc tgcgcccctt |
| 6961 | gacagcatcc acagcctggc tgcctactac atcgactgca tcaggcaggt gcagcccgag |
| 7021 | ggcccctacc gcgtggccgg ctactictac ggggcctgcg tggcctttga aatgtgctcc |
| 7081 | cagctgcagg cccagcagag cccagccccc acccacaaca gcctcttcct gttcgacggc |
| 7141 | tcgcccacct acgtactggc ctacacccag agctaccggg caaagctgac cccaggctgt |
| 7201 | gaggctgagg ctgagacgga ggccatatgc ttcttcgtgc agcagttcac ggacatggag |
| 7261 | cacaacaggg tgctggaggc gctgctgccg ctgaagggcc tagaggagcg tgtggcagcc |
| 7321 | gccgtggacc tgatcatcaa gagccaccag ggcctggacc gccaggagct gagctttgcg |
| 7381 | gcccggtcct tctactacaa gctgcgtgcc gctgagcagt acacacccaa ggccaagtac |
| 7441 | catggcaacg tgatgctact gcgcgccaag acgggtggcg cctacggcga ggacctgggc |
| 7501 | gcggactaca acctctccca ggtatgcgac gggaaagtat ccgtccacgt catcgagggt |
| 7561 | gaccaccgca cgctgctgga gggcagcggc ctggagtcca tcatcagcat catccacagc |
| 7621 | tccctggctg agccacgcgt gagcgtgcgg gagggctagg cccgtgcccc cgcctgccac |
| 7681 | cggaggtcac tccaccatcc ccaccccacc ccaccccacc cccgccatgc aacgggattg |
| 7741 | aagggtcctg ccggtgggac cctgtccggc ccagtgccac tgccccccga ggctgctaga |
| 7801 | tgtaggtgtt aggcatgtcc cacccacccg ccgcctccca cggcacctcg gggacaccag |
| 7861 | agctgccgac ttggagactc ctggtctgtg aagagccggt ggtgcccgtg cccgcaggaa |
| 7921 | ctgggctggg cctcgtgcgc ccgtggggtc tgcgcttggt ctttctgtgc ttggatttgc |
| 7981 | atatttattg cattgctggt agagaccccc aggcctgtcc accctgccaa gactcctcag |
| 8041 | gcagcgtgtg ggtcccgcac tctgccccca tttccccgat gtcccctgcg ggcgcgggca |
| 8101 | gccacccaag cctgctggct gcggccccct ctcggccagg cattggctca gcccgctgag |
| 8161 | tggggggtcg tgggccagtc cccgaggagc tgggcccctg cacaggcaca cagggcccgg |
| 8221 | ccacacccag cggccccccg cacagccacc cgtggggtgc tgcccttatg cccggcgccg |
| 8281 | ggcaccaact ccatgtttgg tgtttgtctg tgtttgtttt tcaagaaatg attcaaattg |
| 8341 | ctgcttggat tttgaaattt actgtaactg tcagtgtaca cgtctggacc ccgtttcatt |
| 8401 | tttacaccaa tttggtaaaa atgctgctct cagcctccca caattaaacc gcatgtgatc |
| 8461 | tcca, |

or a sequence with at least 55%, 60%, 70%, 80%, 90%, 95%, 96%, 97%, 98%, 99% sequence identity thereto (the gene is located at chromosome 17q25.3).

FASN inhibitors can include inhibitors of gene expression (e.g., transcription from genomic DNA or translation from mRNA) or they can include inhibiting the function of the FASN enzyme, such as with antibodies (e.g., monoclonal antibodies), small molecules and inhibitory nucleic acids. Small molecule FASN inhibitors include, but are not limited to, cerulenin (a natural product that irreversibly inhibits fatty acid synthase by covalently binding to the active site), C75 (a synthetic analog of cerulenin that inhibits fatty acid synthase), orlistat (a lipase inhibitor that also inhibits fatty acid synthase), Fasnall (a small molecule inhibitor specifically targeting fatty acid synthase), TVB-2640 (denifanstat; a clinical-stage FASN inhibitor developed by Sagimet Biosciences), TVB-3664 (FASN inhibitor developed by Sagimet Biosciences), TVB-3567 (Sagimet Biosciences), triclosan (a broad-spectrum antimicrobial agent that inhibits fatty acid synthase), GSK837149A (FAS inhibitor), bisamide scaffold ((AstraZeneca); a class of synthetic compounds targeting fatty acid synthase), hydroxyquinoline-2(1H)-one scaffold (Merck; synthetic class of FAS inhibitors), platensimycin (a natural product with potent fatty acid synthase inhibitory activity) or epigallocatechin gallate (EGCG; polyphenol found in green tea that has been shown to inhibit fatty acid synthase).

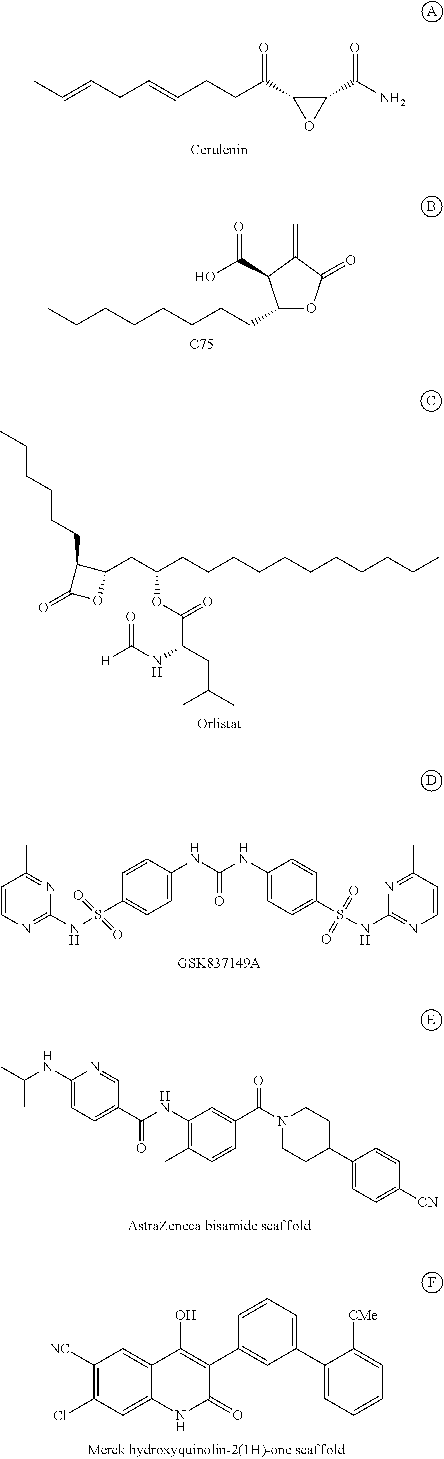

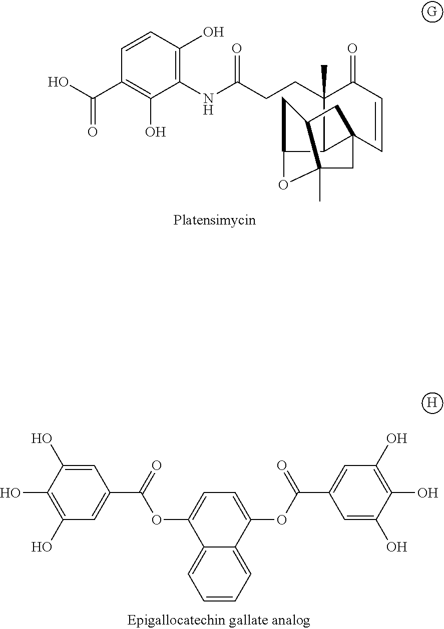

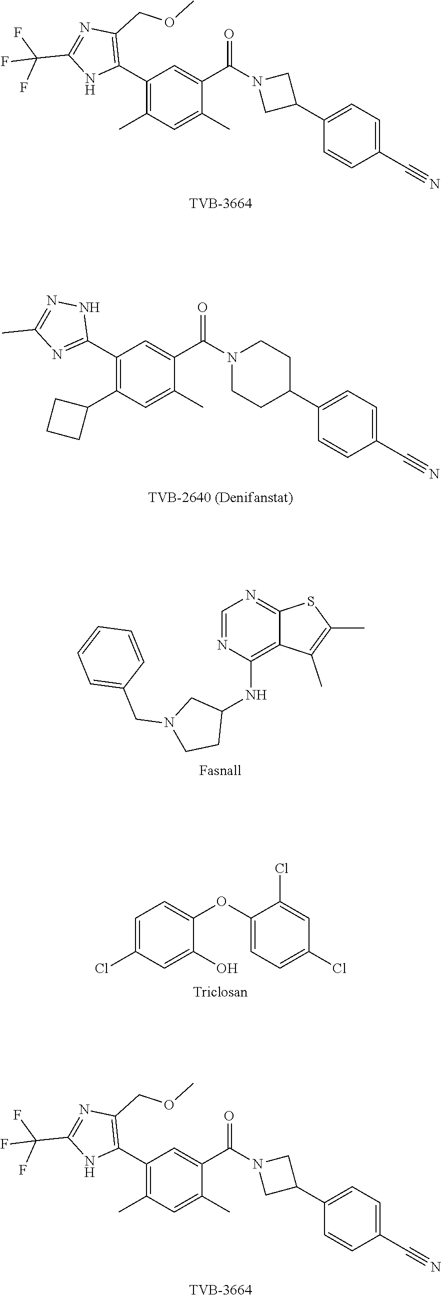 [SMILES: COCc1nc(C(F)(F)F)[nH]c1-c1cc(C(=O)N2CC(c3ccc(C#N)cc3)C2)c(C)cc1C ; Cc1n[nH]c(-c2cc(C(=O)N3CCC(c4ccc(C#N)cc4)CC3)c(C)cc2C2CCC2)n1 ; Cc1sc2ncnc(NC3CCN(Cc4ccccc4)C3)c2c1C ; Oc1cc(Cl)ccc1Oc1ccc(Cl)cc1Cl ; COCc1nc(C(F)(F)F)[nH]c1-c1cc(C(=O)N2CC(c3ccc(C#N)cc3)C2)c(C)cc1C]

### PD-1/PD-L1 Therapy

PD-1 therapy, a type of cancer immunotherapy, works by targeting the PD-1/PD-L1 pathway to boost the immune system's ability to fight cancer. PD-1 and PD-L1 are proteins that can inhibit T-cell activity, preventing them from attacking cancer cells. PD-1 therapy blocks this interaction, allowing T-cells to recognize and destroy cancer cells more effectively.

PD-1 is a receptor on T-cells, and PD-L1 is a protein found on some cancer cells. When PD-1 and PD-L1 interact, they send a signal to T-cells to slow down or stop their activity, effectively “turning off” the immune response against cancer. PD-1 therapy uses drugs called immune checkpoint inhibitors to block the interaction between PD-1 and PD-L1, releasing the brakes on T-cell activity. By blocking this interaction, T-cells are free to attack and destroy cancer cells more effectively, leading to a stronger immune response against the cancer.

### Types of PD-1/PD-L1 Inhibitors:

PD-1/PD-1 inhibitors can include inhibitors of gene expression (e.g., transcription from genomic DNA or translation from mRNA) or they can include inhibiting the function of the PD-I/PD-L1, such with antibodies (e.g., monoclonal antibodies), small molecules and inhibitory nucleic acids.

PD-I inhibitors include, but are not limited to, Pembrolizumab (Keytruda), nivolumab (Opdivo), Cemiplimab (Libtayo), Tislelizumab (Tevimbra), Dostarlimab (Jemperli), Retifanlimab (Zynyz), and/or Toripalimab (logtorzi). PD-L1 inhibitors, include but are not limited to, Atezolizumab (Tecentriq), Avelumab (Bavencio), Cosibelimab (Unloxcyt) and/or Durvalumab (Ipilimumab; Imfinzi).

Exemplary sequence of human PD-1 protein (also known as CD279) can be found at accession number NP_005009.2, the sequence is provided below:

| (SEQ ID NO: 3) | |
|---|---|
| 1 | MQIPQAPWPV VWAVLQLGWR PGWFLDSPDR PWNPPTFSPA LLVVTEGDNA TFTCSFSNTS |
| 61 | ESFVLNWYRM SPSNQTDKLA AFPEDRSQPG QDCRFRVTQL PNGRDFHMSV VRARRNDSGT |
| 121 | YLCGAISLAP KAQIKESLRA ELRVTERRAE VPTAHPSPSP RPAGQFQTLV VGVVGGLLGS |
| 181 | LVLLVWVLAV ICSRAARGTI GARRTGQPLK EDPSAVPVFS VDYGELDFQW REKTPEPPVP |
| 241 | CVPEQTEYAT IVFPSGMGTS SPARRGSADG PRSAQPLRPE DGHCSWPL |

or a sequence with at least 55%, 60%, 70%, 80%, 90%, 95%, 96%, 97%, 98%, 99% sequence identity thereto.

The human PD-1 protein can be coded for by the following mRNA (accession number NM_005018.3):

| (SEQ ID NO: 4) | |
|---|---|
| 1 | gctcacctcc gcctgagcag tggagaaggc ggcactctgg tggggctgct ccaggcatgc |
| 61 | agatcccaca ggcgccctgg ccagtcgtct gggcggtgct acaactgggc tggcggccag |
| 121 | gatggttctt agactcccca gacaggccct ggaacccccc caccttctcc ccagccctgc |
| 181 | tcgtggtgac cgaaggggac aacgccacct tcacctgcag cttctccaac acatcggaga |
| 241 | gcttcgtgct aaactggtac cgcatgagcc ccagcaacca gacggacaag ctggccgcct |
| 301 | tccccgagga ccgcagccag cccggccagg actgccgctt ccgtgtcaca caactgccca |
| 361 | acgggcgtga cttccacatg agcgtggtca gggcccggcg caatgacagc ggcacctacc |
| 421 | tctgtggggc catctccctg gcccccaagg cgcagatcaa agagagcctg cgggcagagc |
| 481 | tcagggtgac agagagaagg gcagaagtgc ccacagccca ccccagcccc tcacccaggc |
| 541 | cagccggcca gttccaaacc ctggtggttg gtgtcgtggg cggcctgctg ggcagcctgg |
| 601 | tgctgctagt ctgggtcctg gccgtcatct gctcccgggc cgcacgaggg acaataggag |
| 661 | ccaggcgcac cggccagccc ctgaaggagg acccctcagc cgtgcctgtg ttctctgtgg |
| 721 | actatgggga gctggatttc cagtggcgag agaagacccc ggagcccccc gtgccctgtg |
| 781 | tccctgagca gacggagtat gccaccattg tctttcctag cggaatgggc acctcatccc |
| 841 | ccgcccgcag gggctcagct gacggccctc ggagtgccca gccactgagg cctgaggatg |
| 901 | gacactgctc ttggcccctc tgaccggctt ccttggccac cagtgttctg cagaccctcc |
| 961 | accatgagcc cgggtcagcg catttcctca ggagaagcag gcagggtgca ggccattgca |
| 1021 | ggccgtccag gggctgagct gcctgggggc gaccggggct ccagcctgca cctgcaccag |
| 1081 | gcacagcccc accacaggac tcatgtctca atgcccacag tgagcccagg cagcaggtgt |
| 1141 | caccgtcccc tacagggagg gccagatgca gtcactgctt caggtcctgc cagcacagag |
| 1201 | ctgcctgcgt ccagctccct gaatctctgc tgctgctgct gctgctgctg ctgctgcctg |
| 1261 | cggcccgggg ctgaaggcgc cgtggccctg cctgacgccc cggagcctcc tgcctgaact |
| 1321 | tgggggctgg ttggagatgg ccttggagca gccaaggtgc ccctggcagt ggcatcccga |
| 1381 | aacgccctgg acgcagggcc caagactggg cacaggagtg ggaggtacat ggggctgggg |
| 1441 | actccccagg agttatctgc tccctgcagg cctagagaag tttcagggaa ggtcagaaga |
| 1501 | gctcctggct gtggtgggca gggcaggaaa cccctccacc tttacacatg cccaggcagc |
| 1561 | acctcaggcc ctttgtgggg cagggaagct gaggcagtaa gcgggcaggc agagctggag |
| 1621 | gcctttcagg cccagccagc actctggcct cctgccgccg cattccaccc cagcccctca |
| 1681 | caccactcgg gagagggaca tcctacggtc ccaaggtcag gagggcaggg ctggggttga |
| 1741 | ctcaggcccc tcccagctgt ggccacctgg gtgttgggag ggcagaagtg caggcaccta |
| 1801 | gggcccccca tgtgcccacc ctgggagctc tccttggaac ccattactga aattatttaa |
| 1861 | aggggttggc cgggctccca ccagggcctg ggtgggaagg tacaggcgtt cccccggggc |
| 1921 | ctagtacccc cgccgtggcc tatccactcc tcacatccac acactgcacc cccactcctg |
| 1981 | gggcagggcc accagcatcc aggcggccag caggcacctg agtggctggg acaagggatc |
| 2041 | ccccttccct gtggttctat tatattataa ttataattaa atatgagagc atgctaa |

or a sequence with at least 55%, 60%, 70%, 80%, 90%, 95%, 96%, 97%, 98%, 99% sequence identity thereto (the gene is located at Chr 2: 241.85-241.86 Mb).

Exemplary sequence of human PD-L1 protein (also known as CD274) can be found at accession number NP_001254635, the sequence is provided below:

| (SEQ ID NO: 5) | |
|---|---|
| 1 | MRIFAVFIFM TYWHLLNAPY NKINQRILVV DPVTSEHELT CQAEGYPKAE VIWTSSDHQV |
| 61 | LSGKTTTTNS KREEKLFNVT STLRINTTIN EIFYCTFRRL DPEENHTAEL VIPELPLAHP |
| 121 | PNERTHLVIL GAILLCLGVA LTFIFRIRKG RMMDVKKCGI QDTNSKKQSD THLEET |

or a sequence with at least 55%, 60%, 70%, 80%, 90%, 95%, 96%, 97%, 98%, 99% sequence identity thereto.

The human PD-L1 protein can be coded for by the following rRNA (accession number NM_014143.4):

| (SEQ ID NO: 6) | |
|---|---|
| 1 | agttctgcgc agcttcccga ggctccgcac cagccgcgct tctgtccgcc tgcagggcat |
| 61 | tccagaaaga tgaggatatt tgctgtcttt atattcatga cctactggca tttgctgaac |
| 121 | gcatttactg tcacggttcc caaggaccta tatgtggtag agtatggtag caatatgaca |
| 181 | attgaatgca aattcccagt agaaaaacaa ttagacctgg ctgcactaat tGtctattgg |
| 241 | gaaatggagg ataagaacat tattcaattt gtgcatggag aggaagacct gaaggttcag |
| 301 | catagtagct acagacagag ggcccggctg ttgaaggacc agctctccct gggaaatgct |
| 361 | gcacttcaga tcacagatgt gaaattgcag gatgcagggg tgtaccgctg catgatcagc |
| 421 | tatggtggtg ccgactacaa gcgaattact gtgaaagtca atgccccata caacaaaatc |
| 481 | aaccaaagaa ttttggttgt ggatccagtc acctctgaac atgaactgac atgtcaggct |
| 541 | gagggctacc ccaaggccga agtcatctgg acaagcagtg accatcaagt cctgagtggt |
| 601 | aagaccacca ccaccaattC caagagagag gagaagcttt tcaatgtgac cagcacactg |
| 661 | agaatcaaca caacaactaa tgagattttc tactgcactt ttaggagatt agatcctgag |
| 721 | gaaaaccata cagctgaatt ggtcatccca gaactacctc tggcacatcc tccaaatgaa |
| 781 | aggactcact tggtaattct gggagccatc ttattatgcc ttggtgtagc actgacattc |
| 841 | atcttccgtt taagaaaagg gagaatgatg gatgtgaaaa aatgtggcat ccaagataca |
| 901 | aactcaaaga agcaaagtga tacacatttg gaggagacgt aatccagcat tggaacttct |
| 961 | gatcttcaag cagggattct caacctgtgg tttaggggtt catcggggct gagcgtgaca |
| 1021 | agaggaagga atgggcccgt gggatgcagg caatgtggga cttaaaaggc ccaagcactg |
| 1081 | aaaatggaac ctggcgaaag cagaggagga gaatgaagaa agatggagtc aaacagggag |
| 1141 | cctggaggga gaccttgata ctttcaaatg cctgaggggc tcatcgacgc ctgtgacagg |
| 1201 | gagaaaggat acttctgaac aaggagcctc caagcaaatc atccattgct catcctagga |
| 1261 | agacgggttg agaatcccta atttgagggt cagttcctgc agaagtgccc tttgcctcca |
| 1321 | ctcaatgcct caatttgttt tctgcatgac tgagagtctc agtgttggaa cgggacagta |
| 1381 | tttatgtatg agtttttcct atttattttg agtctgtgag gtcttcttgt catgtgagtg |
| 1441 | tggttgtgaa tgatttcttt tgaagatata ttgtagtaga tgttacaatt ttgtcgccaa |
| 1501 | actaaacttg ctgcttaatg atttgctcac atctagtaaa acatggagta tttgtaaggt |
| 1561 | gcttggtctc ctctataact acaagtatac attggaagca taaagatcaa accgttggtt |
| 1621 | gcataggatg tcacctttat ttaacccatt aatactctgg ttgacctaat cttattctca |
| 1681 | gacctcaagt gtctgtgcag tatctgttcc atttaaatat cagctttaca attatgtggt |
| 1741 | agcctacaca cataatctca tttcatcgct gtaaccaccc tcttgtgata accactatta |
| 1801 | ttttacccat cgtacagctg aggaagcaaa cagattaagt aacttgccca aaccagtaaa |
| 1861 | tagcagacct cagactgcca cccactgtcc ttttataata caatttacag ctatatttta |
| 1921 | ctttaagcaa ttcttttatt caaaaaccat ttattaagtg cccttgcaat atcaatcgct |
| 1981 | gtgccaggca ttgaatctac agatgtgagc aagacaaagt acctgtcctc aaggagctca |
| 2041 | tagtataatg aggagattaa caagaaaatg tattattaca atttagtcca gtgtcatagc |
| 2101 | ataaggatga tgcgagggga aaacccgagc agtgttgcca agaggaggaa ataggccaat |
| 2161 | gtggtctggg acggttggat atacttaaac atcttaataa tcagagtaat tttcatttac |
| 2221 | aaagagaggt cggtacttaa aataaccctg aaaaataaca ctggaattcc ttttctagca |
| 2281 | ttatatttat tcctgatttg cctttgccat ataatctaat gcttgtttat atagtgtctg |
| 2341 | gtattgttta acagttctgt cttttctatt taaatgccac taaattttaa attcatacct |
| 2401 | ttccatgatt caaaattcaa aagatcccat gggagatggt tggaaaatct ccacttcatc |
| 2461 | ctccaagcca ttcaagtttc ctttccagaa gcaactgcta ctgcctttca ttcatatgtt |
| 2521 | cttctaaaga tagtctacat ttggaaatgt atgttaaaag cacgtatttt taaaattttt |
| 2581 | ttcctaaata gtaacacatt gtatgtctgc tgtgtacttt gctattttta tttattttag |
| 2641 | tgtttcttat atagcagatg gaatgaattt gaagttccca gggctgagga tccatgcctt |
| 2701 | ctttgtttct aagttatctt tcccatagct tttcattatc tttcatatga tccagtatat |
| 2761 | gttaaatatg tcctacatat acatttagac aaccaccatt tgttaagtat ttgctctagg |
| 2821 | acagagtttg gatttgttta tgtttgctca aaaggagacc catgggctct ccagggtgca |
| 2881 | ctgagtcaat ctagtcctaa aaagcaatct tattattaac tctgtatgac agaatcatgt |
| 2941 | ctggaacttt tgttttctgc tttctgtcaa gtataaactt cactttgatg ctgtacttgc |
| 3001 | aaaatcacat tttctttctg gaaattccgg cagtgtacct tgactgctag ctaccctgtg |
| 3061 | ccagaaaagc ctcattcgtt gtgcttgaac ccttgaatgc caccagctgt catcactaca |
| 3121 | cagccctcct aagaggcttc ctggaggttt cgagattcag atgccctggg agatcccaga |
| 3181 | gtttcctttc cctcttggcc atattctggt gtcaatgaca aggagtacct tggctttgcc |
| 3241 | acatgtcaag gctgaagaaa cagtgtctcc aacagagctc cttgtgttat ctgtttgtac |
| 3301 | atgtgcattt gtacagtaat tggtgtgaca gtgttctttg tgtgaattac aggcaagaat |
| 3361 | tgtggctgag caaggcacat agtctactca gtctattcct aagtcctaac tcctccttgt |
| 3421 | ggtgttggat ttgtaaggca ctttatccct tttgtctcat gtttcatcgt aaatggcata |
| 3481 | ggcagagatg atacctaatt ctgcatttga ttgtcacttt ttgtacctgc attaatttaa |
| 3541 | taaaatattc ttatttattt tcttacttgg tacaccagca tgtccatttt cttgtttatt |
| 3601 | ttgtgtttaa taaaatgttc agtttaacat ccca |

or a sequence with at least 55%, 60%, 70%, 80%, 90%, 95%, 96%, 97%, 98%, 99% sequence identity thereto (the gene is located at Chr 9: 5.45-5.47).

### Inhibitory Nucleic Acids

The expression of one or more proteins can be inhibited, for example by use of an inhibitory nucleic acid that specifically recognizes a nucleic acid that encodes the protein.

An inhibitory nucleic acid can have at least one segment that will hybridize to a nucleic acid of interest under intracellular conditions. The inhibitory nucleic acid can reduce expression of nucleic acid of interest. A nucleic acid may hybridize to a genomic DNA, a messenger RNA, or a combination thereof. An inhibitory nucleic acid may be incorporated into a plasmid vector or viral DNA, or it may not be. It may be single stranded or double stranded, circular or linear.

An inhibitory nucleic acid is a polymer of ribose nucleotides or deoxyribose nucleotides having more than 13 nucleotides in length. An inhibitory nucleic acid may include naturally occurring nucleotides; synthetic, modified, or pseudo-nucleotides such as phosphorothiolates; as well as nucleotides having a detectable label such as P³², biotin or digoxigenin. An inhibitory nucleic acid can reduce the expression and/or activity of a nucleic acid interest. Such an inhibitory nucleic acid may be completely complementary to a segment of an endogenous nucleic acid (e.g., an RNA). Alternatively, some variability is permitted in the inhibitory nucleic acid sequences relative to the sequences of interest (e.g., pathway inhibitor). An inhibitory nucleic acid can hybridize to a nucleic acid of interest under intracellular conditions or under stringent hybridization conditions and is sufficiently complementary to inhibit expression of the endogenous nucleic acid of interest. Intracellular conditions refer to conditions such as temperature, pH and salt concentrations typically found inside a cell, e.g., an animal or mammalian cell. One example of such an animal or mammalian cell is a cancer cell. Generally, stringent hybridization conditions are selected to be about 5° C. lower than the thermal melting point (Tm) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to the coding sequence of interest, each separated by a stretch of contiguous nucleotides that are not complementary to adjacent coding sequences, can inhibit the function of one or more nucleic acids. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme or an antisense nucleic acid molecule.

The inhibitory nucleic acid molecule may be single or double stranded (e.g., a small interfering RNA (siRNA)) and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex. Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce translation of a target nucleic acid such that translation of the encoded target polypeptide is reduced. SiRNAs mediate post-transcriptional gene silencing in a sequence-specific manner. See, for example, website at invitrogen.com/site/us/en/home/Products-and-Services/Applications/rnai.html. Once incorporated into an RNA-induced silencing complex, siRNA mediate cleavage of the homologous endogenous mRNA transcript by guiding the complex to the homologous mRNA transcript, which is then cleaved by the complex. The siRNA may be homologous and/or complementary to any region of the target transcript. The region of homology may be 30 or 40 nucleotides or less in length, such less than 25 nucleotides, and such as about 21 to 23 nucleotides in length. SiRNA is typically double stranded and may have two-nucleotide 3′ overhangs, for example, 3′ overhanging UU dinucleotides. Methods for designing siRNAs are known to those skilled in the art. See, for example, Elbashir et al. Nature 411: 494-498 (2001); Harborth et al. Antisense Nucleic Acid Drug Dev. 13: 83-106 (2003).

The pSuppressorNeo vector for expressing hairpin siRNA, commercially available from IMGENEX (San Diego, California), can be used to generate siRNA for inhibiting expression of targets. The construction of the siRNA expression plasmid involves the selection of the target region of the mRNA, which can be a trial-and-error process. However, Elbashir et al. have provided guidelines that appear to work ˜80% of the time. Elbashir, S. M., et al., Analysis of gene function in somatic mammalian cells using small interfering RNAs. Methods, 2002. 26(2): p. 199-213. Accordingly, for synthesis of synthetic siRNA, a target region may be selected about 50 to 100 nucleotides downstream of the start codon. The 5′ and 3′ untranslated regions and regions close to the start codon should be avoided as these may be richer in regulatory protein binding sites. As siRNA can begin with AA, have 3′ UU overhangs for both the sense and antisense siRNA strands, and have an approximate 50% G/C content. An example of a sequence for a synthetic siRNA is 5′-AA(N19)UU, where N is any nucleotide in the mRNA sequence and should be approximately 50% G-C content. The selected sequence(s) can be compared to others in the human genome database to minimize homology to other known coding sequences (e.g., by Blast search, for example, through the NCBI website).

siRNAs may be chemically synthesized, created by in vitro transcription, or expressed from an siRNA expression vector or a PCR expression cassette. See, e.g., website at invitrogen.com/site/us/en/home/Products-and-Services/Applications/rnai.html. When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, preferably, 3 to 23 nucleotides in length, and may be of various nucleotide sequences including, AUG, CCC, UUCG, CCACC, CTCGAG, AAGCUU, CCACACC and UUCAAGAGA. SiRNAs also may be produced in vivo by cleavage of double-stranded RNA introduced directly or via a transgene or virus. Amplification by an RNA-dependent RNA polymerase may occur in some organisms.

An inhibitory nucleic acid such as a short hairpin RNA siRNA or an antisense oligonucleotide may be prepared using methods such as by expression from an expression vector or expression cassette that includes the sequence of the inhibitory nucleic acid. Alternatively, it may be prepared by chemical synthesis using naturally occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target nucleic acids.

An inhibitory nucleic acid may be prepared using available methods, for example, by expression from an expression vector encoding a complementarity sequence of the nucleic acids described herein. Alternatively, it may be prepared by chemical synthesis using naturally occurring nucleotides, modified nucleotides or any mixture of combination thereof. In some embodiments, the nucleic acids described herein are made from modified nucleotides or non-phosphodiester bonds, for example, that are designed to increase biological stability of the nucleic acids or to increase intracellular stability of the duplex formed between the inhibitory nucleic acids and other (e.g., endogenous) nucleic acids.

For example, nucleic acids can be peptide nucleic acids that have peptide bonds rather than phosphodiester bonds.

Naturally occurring nucleotides that can be employed in the nucleic acids include the ribose or deoxyribose nucleotides adenosine, guanine, cytosine, thymine and uracil. Examples of modified nucleotides that can be employed in the nucleic acids include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-IN-2-carboxypropyl) uracil, (acp3)w, and 2,6-diaminopurine.

Thus, inhibitory nucleic acids described herein may include modified nucleotides, as well as natural nucleotides such as combinations of ribose and deoxyribose nucleotides. The inhibitory nucleic acids and may be of same length as wild type. The inhibitory nucleic acids described herein can also be longer and include other useful sequences. In some embodiments, the inhibitory nucleic acids described herein are somewhat shorter. For example, inhibitory nucleic acids described herein can include a segment that has a nucleic acid sequence that can be missing up to 5 nucleotides, or missing up to 10 nucleotides, or missing up to 20 nucleotides, or missing up to 30 nucleotides, or missing up to 50 nucleotides, or missing up to 100 nucleotides from the 5′ or 3′ end.

### Radiation

Radiation therapy or radiotherapy (RT, RTx, or XRT) is a treatment using ionizing radiation, generally provided as part of cancer therapy to either kill or control the growth of malignant cells. Radiation therapy may be curative in a number of types of cancer if they are localized to one area of the body and have not spread to other parts. It may also be used as part of adjuvant therapy, to prevent tumor recurrence after surgery to remove a primary malignant tumor (for example, early stages of breast cancer). Radiation therapy is synergistic with chemotherapy, and has been used before, during, and after chemotherapy in susceptible cancers.

Examples of cancer radiation treatments, include, but are not limited to, External Beam Radiation Therapy (EBRT or XRT) (e.g., Intensity-Modulated Radiation Therapy (IMRT), Image-Guided Radiation Therapy (IGRT), Stereotactic Body Radiation Therapy (SBRT), and/or Stereotactic Radiosurgery (SRS)), Proton Therapy (e.g., Proton Beam Therapy), Intensity-modulated radiation therapy (IMRT), Volumetric modulated arc therapy (VMAT), Temporally feathered radiation therapy (TFRT), Brachytherapy (Internal Radiation; Low-Dose Rate (LDR) Brachytherapy and or High-Dose Rate (HDR) Brachytherapy), Systemic Radiation Therapy (e.g., Radioisotope Therapy, Iodine-131, Radium-223 (Xofigo), and/or Lutetium-177 (Lu-177)), Targeted Radiation Therapy (e.g., Theranostics, Peptide Receptor Radionuclide Therapy (PRRT), gamma knife).

### Chemotherapy

Chemotherapy (often abbreviated chemo, sometimes CTX and CTx) is the type of cancer treatment that uses one or more anti-cancer drugs (chemotherapeutic agents or alkylating agents) in a standard regimen. Chemotherapy may be given with a curative intent (which almost always involves combinations of drugs), or it may aim only to prolong life or to reduce symptoms (palliative chemotherapy).

Chemotherapy involves various drug types to combat cancer. Common chemotherapy drugs include alkylating agents like Busulfan, Carboplatin, and Cisplatin, as well as antimetabolites like 5-FU, Cytarabine, and Methotrexate. Mitotic inhibitors such as Paclitaxel and Vinblastine are also used.

### Common Chemotherapy Drug Categories:

Alkylating Agents: These drugs damage cancer cells' DNA, interfering with their ability to grow and divide. Examples include Busulfan, Carboplatin, and Cisplatin.

Antimetabolites: These drugs act as false building blocks for DNA, disrupting its synthesis. Examples include 5-FU, Cytarabine, and Methotrexate.

Mitotic Inhibitors: These drugs interfere with the process of cell division, preventing cancer cells from replicating. Examples include Paclitaxel and Vinblastine.

Topoisomerase Inhibitors: These drugs disrupt the enzyme responsible for DNA structure, causing DNA strand breaks. Examples include Irinotecan and Etoposide.

Hormonal Therapies: These drugs interfere with hormones that stimulate cancer growth. Examples include Tamoxifen and Anastrozole.

Other Chemotherapy Drugs: Doxorubicin (Adriamycin), Bleomycin (Blenoxane), Cyclophosphamide (Cytoxan), Lomustine (CeeNU), Docetaxel (Taxotere), Irinotecan (Camptosar), Etoposide, and Ifosfamide.

### Compositions

The invention also relates to compositions containing one or more active agents. Such active agents can be a polypeptide, a nucleic acid encoding a polypeptide (e.g., within an expression cassette or expression vector), a modified cell, an inhibitory nucleic acid, a small molecule, an antibody, or a combination thereof. The compositions can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof. For example, the compositions can comprise a combination of at least one FAS inhibitor and at least one inhibitor of PD-I and/or PD-L1.

The composition can be formulated in any convenient form.

In some embodiments, the active agents of the invention (e.g., polypeptide, a nucleic acid encoding a polypeptide (e.g., within an expression cassette or expression vector), an antibody, an inhibitory nucleic acid, a small molecule, modified cells, or a combination thereof), are administered in a “therapeutically effective amount.” Such a therapeutically effective amount is an amount sufficient to obtain the desired physiological effect, such a reduction of at least one symptom of disease.

For example, active agents can reduce the symptoms of disease by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%.

To achieve the desired effect(s), the active agents may be administered as single or divided dosages. For example, active agents can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the type of small molecules, compounds, peptides, or nucleic acid chosen for administration, the disease, the weight, the physical condition, the health, and the age of the mammal. Such factors can be readily determined by the clinician employing animal models or other test systems that are available in the art.

Administration of the active agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the active agents and compositions of the invention may be essentially continuous over a preselected period of time or may be in a series of spaced doses. Both local and systemic administration is contemplated.

To prepare the composition, small molecules, compounds, polypeptides, nucleic acids, expression cassettes, and other agents are synthesized or otherwise obtained, purified as necessary or desired. These small molecules, compounds, polypeptides, nucleic acids, expression cassettes, and other agents can be suspended in a pharmaceutically acceptable carrier and/or lyophilized or otherwise stabilized. The small molecules, compounds, polypeptides, nucleic acids, expression cassettes, other agents, and combinations thereof can be adjusted to an appropriate concentration, and optionally combined with other agents. The absolute weight of a given small molecule, compound, polypeptide, nucleic acid, ribonucleoprotein complex, and/or other agents included in a unit dose can vary widely. For example, about 0.01 to about 2 g, or about 0.1 to about 500 mg, of at least one molecule, compound, polypeptide, nucleic acid, and/or other agents, or a plurality of molecules, compounds, polypeptides, nucleic acids, and/or other agents can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the active agents of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

It will be appreciated that the amount of active agent for use in treatment will vary not only with the particular carrier selected but also with the route of administration, the nature of the condition being treated and the age and condition of the patient. Ultimately the attendant health care provider can determine proper dosage. In addition, a pharmaceutical composition can be formulated as a single unit dosage form.

Thus, one or more suitable unit dosage forms comprising the active agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The active agent(s) may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the active agent with liquid carriers, solid matrices, semi-solid carriers, finely divided solid carriers or combinations thereof, and then, if necessary, introducing or shaping the product into the desired delivery system. For example, the active agent(s) can be linked to a convenient carrier such as a nanoparticle, albumin, polyalkylene glycol, or be supplied in prodrug form. The active agent(s), and combinations thereof can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of inhibitors can also involve parenteral or local administration of the in an aqueous solution or sustained release vehicle.

While the active agent(s) and/or other agents can sometimes be administered in an oral dosage form, that oral dosage form can be formulated so as to protect the small molecules, compounds, polypeptides, nucleic acids, expression cassettes, and combinations thereof from degradation or breakdown before the small molecules, compounds, polypeptides (including antibodies), nucleic acids encoding such polypeptides, expression cassettes, and combinations thereof provide therapeutic utility. For example, in some cases the small molecules, compounds, polypeptides, nucleic acids encoding such polypeptide, expression cassettes, and/or other agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The active agent(s) and/or other agents can be formulated in dry form (e.g., in freeze-dried form), in the presence or absence of a carrier. If a carrier is desired, the carrier can be included in the pharmaceutical formulation, or can be separately packaged in a separate container, for addition to the inhibitor that is packaged in dry form, in suspension or in soluble concentrated form in a convenient liquid.

An active agent(s) and/or other agents can be formulated for parenteral administration (e.g., by injection, for example, bolus injection or continuous infusion) and may be presented in unit dosage form in ampoules, prefilled syringes, small volume infusion containers or multi-dose containers with an added preservative.

The present description is further illustrated by the following example, which should not be construed as limiting in any way. The contents of all cited references (including literature references, issued patents, published patent applications as cited throughout this application) are hereby expressly incorporated by reference.

### Example

As mortality from GBM is predicted to increase, new treatment strategies are needed. Radiation therapy (RT), a therapeutic modality for GBM, is still considered standard of care in combination with temozolomide (TMZ) (Stupp et al., N Engl J Med 2005). Although RT exhibits immunogenic properties in some cancers, the fact that GBM invariably recurs (Milano et al., Int J Radiat Oncol Biol Phys 2010; Ogura et al., Radiat Oncol 2013) suggests that RT does not stimulate long-lasting immunity in this disease. The mechanisms responsible for immune escape of irradiated GBM are yet unknown.

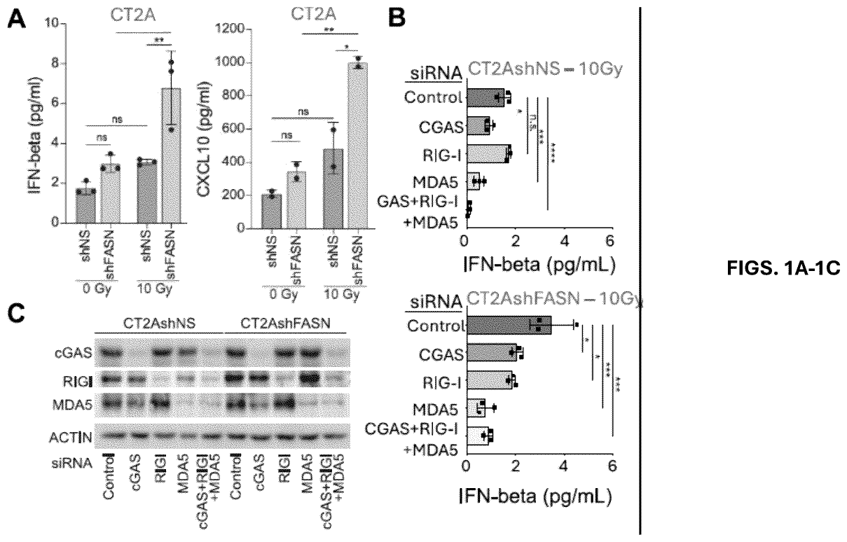

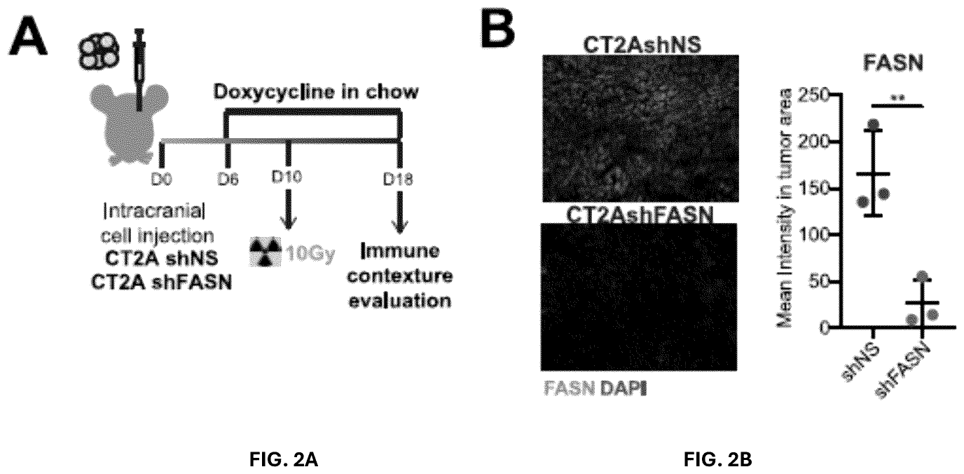

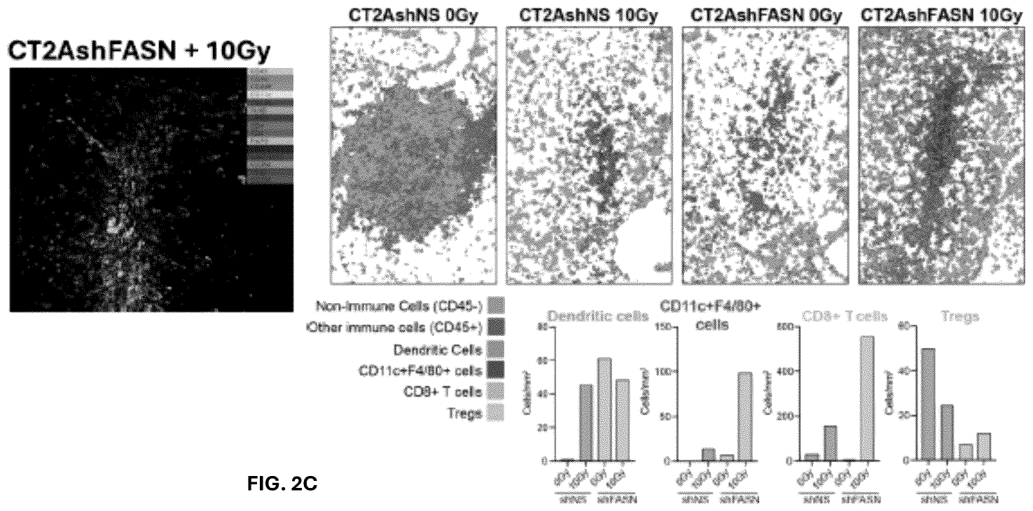

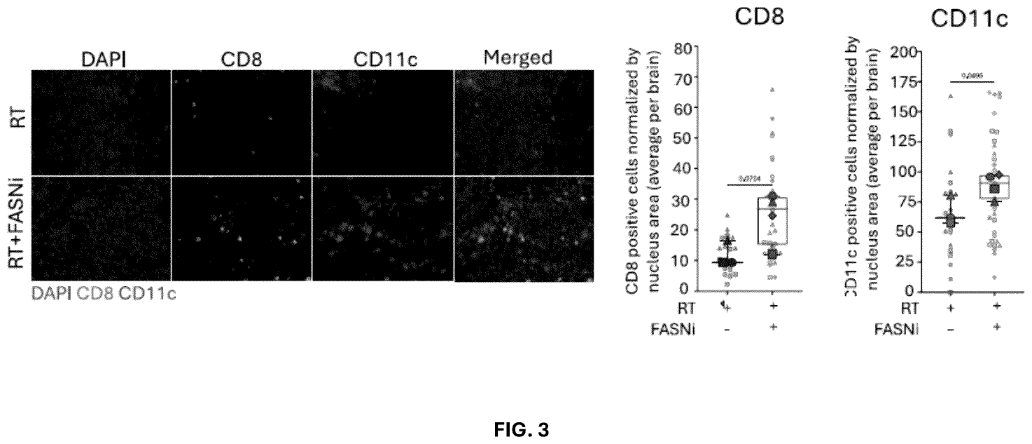

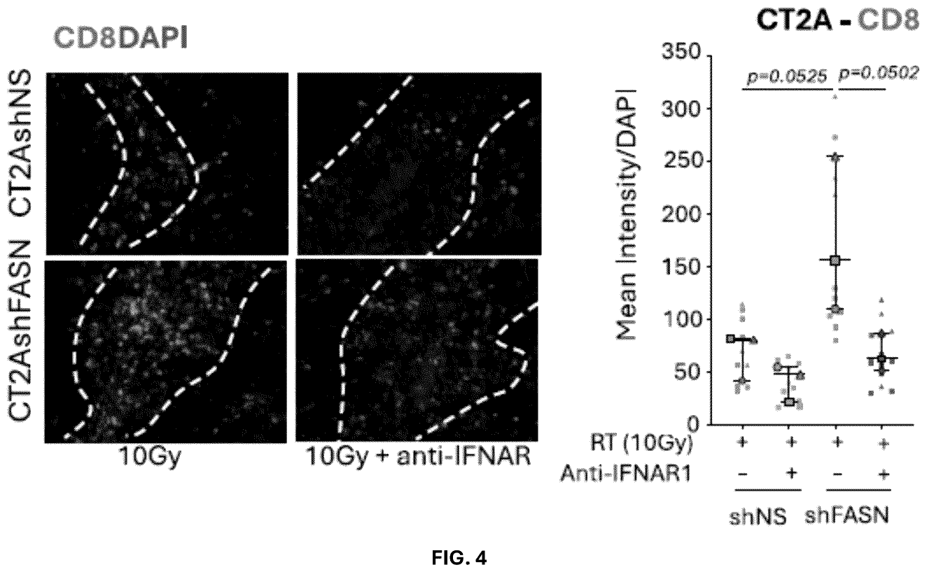

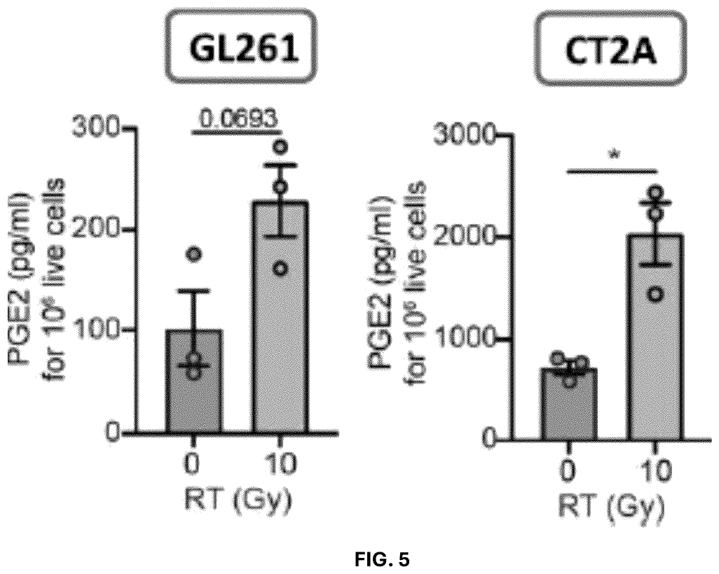

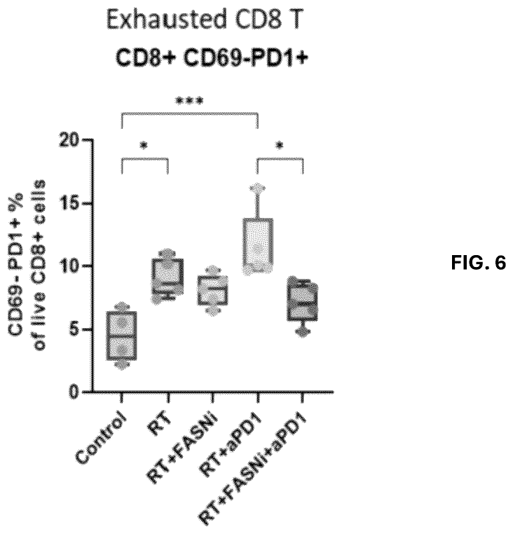

Data show that RT drives GBM resistance by generating a lipogenic environment permissive to GBM survival. Specifically, the biosynthesis of fatty acids (FAs) was identified in vitro and in vivo as a dominant metabolic pathway that protects GBM from undergoing endoplasmic reticulum (ER) stress, thereby permitting the escape of RT-induced apoptosis (De Martino et al. Cancer Letters 2023; publication attached). It was found that irradiated GBM increases the accumulation of arachidonic acid (AA), an unsaturated FA that is upstream of the synthesis of eicosanoids and prostanoids (i.e., the prostaglandin family) (Johnson et al., Front Pharmacol 2020; Wang et al., Signal Transduct Target Ther 2021). While lipid metabolism has been previously targeted as a strategy to radiosensitize tumors (Gupta et al. Front Oncol 2020; Rae et al., Adv Radiat Oncol 2020), no study has investigated the impact of FAs on RT-induced anti-tumor immunity in GBM. The data provided herein show, for the first time, that RT induces the fatty acid synthase (FASN) to generate FAs that permit GBM cells to escape from CD8 cytotoxic T lymphocytes (CTLs). Specifically, it was found that FAs preserve the integrity of mitochondria (FIG. 6A from De Martino et al. Cancer Letters 2023 attached) and impair cancer cell-intrinsic IFN-I mediated by cGAS, RIG-I, and MDA5 (FIG. 1), a step to jumpstart anti-tumor immunity (Vanpouille-Box et al., Nat Comm 2017). In line with these data, it was demonstrated that FASN blockade in GBM promotes the recruitment of CTLs in irradiated GBM mice (FIGS. 2 and 3), an effect that was abrogated when IFN-I response was blocked with an anti-IFN alpha/beta receptor subunit 1 (IFNAR1) monoclonal antibody (FIG. 4). It was found that FAs production after RT increases the release of prostaglandin E2 (FIG. 5), a potent immunosuppressive cytokine capable of promoting PD-1/PD-L1 (Prima, PNAS, 2017; Goto et al., Front Vet Sci 2020), thus showing that FASN blockade can sensitize irradiated GBM to anti-PD-1 therapy. Supporting this concept, the data reveal that concomitant blockade of FASN and PD-1 significantly decreased exhausted CD8+ T cells in irradiated GBM (FIG. 6) and led to 85% of mice surviving beyond 100 days (FIG. 7B). Confirming the induction of potent anti-tumor immunity against irradiated GBM, rechallenge of the surviving mice with a fresh GBM cell inoculum revealed that mice treated with RT, FASN blockade and anti-PD-1 developed immune memory (FIG. 7C).

Overall, the data show that RT is reprogramming the energy metabolism of GBM towards the synthesis of lipids as an immune resistance mechanism. Consequently, the combination of FASN inhibition, anti-PD-1 and RT represent a novel intervention to improve survival of GBM patients. This is the first time that this combination strategy has been disclosed to treat glioblastoma.

What is claimed is:

1. A method to treat cancer in a subject comprising administering to a subject in need thereof an effective amount of a fatty acid synthase (FASN) inhibitor and an effective amount of a programmed cell death protein 1 (PD-1) inhibitor, a programmed death ligand 1 (PD-L1) inhibitor or combination thereof, wherein the subject is or will be administered radiation therapy (RT) and/or chemotherapy.

2. The method of claim 1, wherein the subject is administered the FASN inhibitor, PD-1 inhibitor and/or PD-L1 inhibitor one or more days prior to administration of radiation and/or chemotherapy.

3. The method of claim 2, wherein the administration of the FASN inhibitor, PD-1 inhibitor and/or PD-L1 inhibitor continues concurrently with and optionally following radiation therapy and/or chemotherapy.

4. The method of claim 1, wherein the FASN inhibitor is administered prior to, during and after RT and wherein PD-1 inhibitor and/or PD-L1 inhibitor is administered after RT.

5. The method of claim 1, wherein the subject has prolonged survival with treatment with the FASN inhibitor, PD-1 inhibitor and/or PD-L1 inhibitor as compared to survival without such treatment.

6. The method of claim 1, wherein the RT is selected from external beam radiation therapy (EBRT), proton therapy, systemic radiation therapy, targeted radiation therapy or a combination thereof.

7. The method of claim 1, wherein the chemotherapy is selected from alkylating agents, antimetabolites, mitotic inhibitors, topoisomerase inhibitors, hormonal therapies, immune therapies or a combination thereof.

8. The method of claim 1, wherein the PD-1 and/or PD-L1 inhibitor is selected from antibodies, small molecules, inhibitory nucleic acids or a combination thereof.

9. The method of claim 8, wherein the PD-1 inhibitor is selected from pembrolizumab, nivolumab, cemiplimab, tislelizumab, dostarlimab, retifanlimab, toripalimab or a combination thereof.

10. The method of claim 8, wherein the PD-L1 inhibitor is selected from atezolizumab, avelumab, cosibelimab, durvalumab or a combination thereof.

11. The method of claim 1, wherein the FASN inhibitor is selected from antibodies, small molecules, inhibitory nucleic acids or a combination thereof.

12. The method of claim 11, wherein the FASN inhibitor is selected from cerulenin, C75, orlistat, Fasnall, TVB-2640, TVB-3664, TVB-3567, triclosan, GSK837149A, bisamide scaffold, hydroxyquinoline-2(1H)-one scaffold, platensimycin, epigallocatechin gallate (EGCG) or combination thereof.

13. The method of claim 1, wherein the cancer has an increased lipid content after RT or chemotherapy.

14. The method of claim 1, wherein the cancer is selected from melanoma, prostate cancer, breast cancer, bladder cancer, lung cancer, glioma and diffuse midline glioma.

15. A kit for treating cancer in a subject, comprising:
a first pharmaceutical composition comprising a programmed cell death protein 1 (PD-1) inhibitor and/or a programmed death ligand 1 (PD-L1) inhibitor;
a second pharmaceutical composition comprising a fatty acid synthase (FASN) inhibitor; and
instructions for administering radiation therapy and/or chemotherapy to said subject in combination with said first pharmaceutical composition and said second pharmaceutical composition.
